SEQUENCE LISTING (2 sequences)

SEQ ID NO: 1 - DNA, 24 bp, Artificial Sequence
  Synthetic Polynucleotide
tttttttttttttttttttttttt

SEQ ID NO: 2 - DNA, 24 bp, Artificial Sequence
  Synthetic Polynucleotide
tttttttttttttttttttttttt

(19) United States
(12) Patent Application Publication
(10) Pub. No.: US 2012/0269730 A1
(43) Pub. Date: Oct. 25, 2012

(54) Intracellular Delivery of Contrast Agents with Functionalized Nanoparticles

(75) Inventors: Chad A. Mirkin, Wilmette, IL (US); Thomas J. Meade, Wilmette, IL (US); Ying Song, Cambridge, MA (US); Xiaoyang Xu, Cambridge, MA (US)

(73) Assignee: Northwestern University, Evanston, IL (US)

(21) Appl. No.: 13/388,629

(22) PCT Filed: Aug. 9, 2010
(86) PCT No.: PCT/US10/44844

§ 371 Date: Jul. 13, 2012

Related U.S. Application Data
(60) Provisional application No. 61/232,300, filed on Aug. 7, 2009.
(60) Provisional application No. 61/239,133, filed on Sep. 2, 2009.

(51) Int. Cl. C07H 21/04; C12Q 1/02; A61K 49/00; B82Y 15/00

(52) U.S. Cl. 424/9.1; 536/24.31; 435/29; 977/773; 977/774; 977/902

(57) ABSTRACT

The present invention is directed to compositions and methods for intracellular delivery of a contrast agent with a functionalized nanoparticle.

### CROSS-REFERENCE TO RELATED APPLICATIONS

This application claims the priority benefit under 35 U.S.C. §119(e) of U.S. Provisional Application No. 61/232,300, filed Aug. 7, 2009, and U.S. Provisional Application No. 61/239,133, filed Sep. 2, 2009, the disclosures of which are incorporated herein by reference in their entirety.

### STATEMENT OF GOVERNMENT INTEREST

This invention was made with government support under Grant Number 5 R01 EB005866-04, awarded by the National Institutes of Health (NIH), and Grant Number 5 U54 CA119341 awarded by the NIH(NCI). The government has certain rights in the invention.

### FIELD OF THE INVENTION

The present invention is directed to compositions and methods for intracellular delivery of a contrast agent with a functionalized nanoparticle.

### BACKGROUND OF THE INVENTION

During the past two decades, magnetic resonance imaging (MRI) has become a powerful technique in clinical diagnosis and biological molecular imaging [Merbach et al., Editors, The Chemistry of Contrast Agents in Medical Magnetic Resonance Imaging, 1st ed., Wiley, New York, 2001; Aime et al., J. Magn. Reson. Imaging 16: 394 (2002); Hu et al., Annu. Rev. Biomed. Eng. 6: 157(2004); Winter et al., Curr. Cardiol. Rep. 8: 65 (2006)]. A significant advantage of MRI is the ability to acquire tomographic information of whole animals with high spatial resolution and soft tissue contrast. In addition, images are acquired without the use of ionizing radiation (e.g., X-ray and CT) or radiotracers (e.g., PET and SPECT) permitting long term longitudinal studies. Since spatial resolution increases with magnetic field strength, the ability to track small cell populations has been realized.

MRI contrast agents are frequently utilized to permit the visual differentiation of cells and tissues that are magnetically similar but histologically distinct. Paramagnetic gadolinium [Gd(III)] complexes are the most widely used contrast agents, as Gd(III) reduces the longitudinal relaxation time (T₁) of local water protons due to its high magnetic moment and symmetric 5-state. Areas enriched with Gd(III) exhibit an increase in signal intensity and appear bright in T₁-weighted images. Furthermore, chelation of the Gd(III) ion (required to decrease latent toxicity) provides a means for chemical modification with targeting or bioactive moieties and cell transduction domains.

Recent advances in design and amplification strategies have produced a wide variety of bioactivatable contrast agents for investigating biologically important events such as ion fluctuation, enzyme activity, peroxide evolution, and temperature variation [Caravan, Chem. Soc. Rev. 35: 512 (2006); Major et al., Acc. Chem. Res. 42: 893 (2009); Aime et al., Acc. Chem. Res. 42: 822 (2009); Duimstra et al., J. Am. Chem. Soc. 127: 12847 (2005); Major et al., Proc. Natl. Acad. Sci. U.S.A. 104: 13881 (2007); Li et al., J. Am. Chem. Soc. 121: 1413 (1999); Caravan et al., J. Am. Chem. Soc. 124: 3152 (2002); Kalman et al., Inorg. Chem. 46: 5260 (2007)]. However, the majority of these agents are incapable of penetrating cells and therefore are of limited use in molecular imaging and cell tracking experiments.

Recent results suggest that Gd(III) contrast agents have shown promise in cell tracking and fate-mapping experiments. For example, tracking stem cells in adult rat brains post stroke and monitoring β-islet cell transplantation has demonstrated potential [Modo et al., Neuroimage 21: 311 (2004); Modo et al., Editors, Molecular and Cellular MR Imaging, CRC Press, FL, 2007; Biancone et al., NMR in biomedicine 20: 40 (2007)]. However, there are few examples of magnetic resonance (MR) probes with the essential characteristics of high Gd(III) loading for enhanced contrast coupled with facile cell uptake and long-term cell retention.

### SUMMARY OF THE INVENTION

Described herein is a nanoparticle composition comprising a nanoparticle functionalized with a polynucleotide, wherein the polynucleotide is conjugated to a contrast agent through a conjugation site. The compositions provided by the present disclosure are useful for delivering a contrast agent based on polynucleotide functionalized nanoparticles (PN-NPs) for cell imaging.

In some embodiments, the contrast agent is a paramagnetic compound and in a specific aspect of this embodiment, the paramagnetic compound is a paramagnetic gadolinium [Gd(III)] complex or a manganese chelate. In a specific embodiment, the manganese chelate is Mn-DPDP.

The disclosure contemplates a polynucleotide functionalized on the nanoparticle wherein the polynucleotide is a homopolymer. In various aspects, the homopolymer is a sequence of thymidine (polyT) nucleotides or the homopolymer is a sequence of uridine (polyU) nucleotides. In certain embodiments, the polynucleotide further comprises a detectable marker and in some aspects, the detectable marker is a fluorophore, a luminophore or an isotope.

In some embodiments, the polynucleotide comprises about 5 nucleotides to about 100 or about 10 nucleotides to about 50 nucleotides. In a specific aspect, the polynucleotide comprises about 15 nucleotides.

The invention further provides a polynucleotide functionalized on the nanoparticle wherein the polynucleotide comprises one to about ten conjugation sites. In one aspect, the polynucleotide comprises five conjugation sites.

The nanoparticle, in some embodiments, comprises about 10 to about 25000 functionalized polynucleotides and in other embodiments, about 50 to about 10000 functionalized polynucleotides, while in further embodiments, about 200 to about 5000 functionalized polynucleotides.

The composition provided, in some embodiments, comprises about 50 to about 2.5×10⁶ contrast agents or about 500 to about 1×10⁶ contrast agents. In various aspects, all of the contrast agents in the composition are the same, and in other aspects, at least two different contrast agents are in the composition.

Compositions contemplated by the present disclosure, in some embodiments, optionally comprise a therapeutic agent.

Also provided by the disclosure is a method of delivering a contrast agent to a cell comprising contacting the cell with a composition as described herein under conditions sufficient to deliver the contrast agent to the cell. In some aspects, the contrast agent is delivered more than once. The methods provided further optionally comprise the step of detecting the contrast agent. In some aspects, the contrast agent is detected by detecting the detectable marker if present.

In some embodiments, the methods provided are part of an imaging procedure. In some aspects, the imaging procedure is selected from the group consisting of magnetic resonance imaging (MRI), computed tomography (CT), X-ray attenuation, luminescence, near infrared spectroscopy, positron emission tomography (PET) and fluorescence.

Methods according to the present disclosure are also provided for delivering a composition as described herein to a cell comprising the step of contacting the cell with a composition provided under conditions to deliver the composition to the cell. Methods of this type optionally include the step of identifying the cell to which the composition has been delivered. Methods provided also optionally include the step of isolating the cell that is identified, and in other aspect, method optionally include the step of administering the isolated cell to a patient in need thereof. In some embodiments, the cell is selected from the group consisting of a cancer cell, a stem cell, a T-cell, and a β-islet cell. Methods wherein delivery is in vivo or in vitro are contemplated. In some aspects, delivery is through intravenous administration, intraarterial administration or both.

In some aspects of the methods provided, delivering a composition of the present disclosure results in increased cellular uptake of the contrast agent relative to its uptake without the contrast agent being associated with the nanoparticle. The present disclosure contemplates, in some aspects, that the uptake is increased about 2-fold to about 100-fold. In further aspects, the uptake is increased about 5-fold to about 5000-fold. In some aspects, the uptake is increased about 10-fold to about 40-fold. In still further aspects, the uptake is increased about 20-fold, and in yet further aspects, the uptake is increased about 50-fold.

In further aspects of the methods provided herein, the relaxivity of the contrast agent is increased relative to the relaxivity of the contrast agent in the absence of being associated with the nanoparticle. In some embodiments, the increase is about 1-fold to about 20-fold. In further embodiments, the increase is about 2-fold fold to about 10-fold, and in a further embodiment the increase is about 3-fold.

In some aspects, delivery of a composition of the disclosure further comprises delivery of an embolic agent. In some embodiments, the embolic agent is selected from the group consisting of a lipid emulsion, a gelatin sponge, a tris acetyl gelatin microsphere, an embolization coil, ethanol, a small molecule drug, a biodegradable microsphere, a non-biodegradable microsphere or polymer, and a self-assemblying embolic material.

The present disclosure additionally provides a kit comprising a composition as disclosed herein.

### DESCRIPTION OF THE DRAWINGS

FIG. 1 depicts time dependent cellular uptake of DNA-Gd(III)-AuNPs compared to DOTA-Gd(III) in NIH/3T3 and HeLa cells. Cells were incubated with 6.5 μM Gd(III) for both contrast agents. Error bars represent ±1 standard deviation of the mean for duplicate experiments.

FIG. 2 depicts concentration dependent cellular uptake of DNA-Gd(III)-AuNPs compared to DOTA-Gd(III) in NIH/3T3 and HeLa cells. Cells were incubated for 24 hours for both contrast agents. Error bars represent ±1 standard deviation of the mean for duplicate experiments.

FIG. 3 depicts a T₁-weighted MR image of NIH/3T3 cells incubated with 20 μM and 5.0 μM [Gd(III) concentrations] DNA-Gd(III)-AuNP and DOTA-Gd(III) for 24 hours at 14.1T and 25° C. (TE=10.2 ms, TR=750 ms, FOV=10×10 mm2, slice thickness=1.0 mm).

### DETAILED DESCRIPTION OF THE INVENTION

The present disclosure provides a composition comprising a PN-NP conjugated to a contrast agent. This conjugate takes advantage of high cellular uptake, excellent stability, and high contrast agent loading of PN-NPs [Rosi et al., Science (Washington, D.C., U.S.) 312: 1027 (2006); Seferos et al., Nano Lett. 9: 308 (2009)]. These are properties not shared by all nanostructures and are a result of the dense loading of the polynucleotides on the surface of the NPs and their ability to bind to proteins, which facilitates endocytosis [Rosi et al., Chem. Rev. 105: 1547 (2005); Giljohann et at, Nano Lett. 7: 3818 (2007); Park et al., Bioorg. Med. Chem. Lett. 18: 6135 (2008); Debouttiere et al., Adv. Funct. Mater. 16: 2330 (2006); Moriggi et al., J. Am. Chem. Soc. 131: 10828 (2009)]. In addition to gene regulation, PN-NPs have been used in detection systems for DNA, proteins, metal ions, small molecules, and intracellular siRNA [Rosi et al., Chem. Rev. 105: 1547 (2005); Mirkin et al., Nature 382: 607 (1996); Elghanian et al., Science 277: 1078 (1997); Taton et al., Science (Washington, D.C.) 289: 1757 (2000); Cao et al., J. Am. Chem. Soc. 125: 14676 (2003); Han et al., J. Am. Chem. Soc. 128: 4954 (2006); Lee et al., Angew. Chem., Int. Ed. 46: 4093 (2007); Xu et al., Angew. Chem., Int. Ed. 46: 3468 (2007); Xu et al., Anal. Chem. 79: 6650 (2007); Giljohann et al., J. Am. Chem. Soc. 131: 2072 (2009); Bowman et al., J. Am. Chem. Soc. 130: 6896 (2008); Liu et al., Angew. Chem., Int. Ed. 46: 7587 (2007); Agasti et al., J. Am. Chem. Soc. 131: 5728 (2009)].

The PN-NP conjugates provided represent a new class of MR contrast agent with the capability of highly efficient cell penetration and accumulation that provides sufficient contrast enhancement for imaging small cell populations with viM contrast agent incubation concentrations. Moreover, these conjugates are optionally labeled with a fluorescent dye permitting multimodal imaging to confirm cell uptake and intracellular accumulation, and providing a means for histological validation [Frullano et al., J. Biol. Inorg. Chem. 12: 939 (2007)].

Accordingly, in some embodiments the present disclosure provides a composition comprising a nanoparticle functionalized with a polynucleotide, wherein the polynucleotide is conjugated to a contrast agent through a conjugation site. Throughout the disclosure, the term “functionalized” is used interchangeably with the terms “attached” and “bound.” As used herein, a “conjugation site” is understood to mean a site on a polynucleotide to which a contrast agent is attached.

### Nanoparticles

Compositions of the present disclosure comprise nanoparticles as described herein. Nanoparticles are provided which are functionalized to have a polynucleotide attached thereto. The size, shape and chemical composition of the nanoparticles contribute to the properties of the resulting PN-NP. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical particles), anisotropic particles (such as non-spherical rods, tetrahedral, and/or prisms) and core-shell particles, such as those described in U.S. Pat. No. 7,238,472 and International Publication No. WO 2003/08539, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials.

Also, as described in U.S. Patent Publication No 2003/0147966, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., HaVashi, Vac. Sci. Technol. A5(4):1375-84 (1987); Hayashi, Physics Today, 44-60 (1987); MRS Bulletin, January 1990, 16-47. As further described in U.S. Patent Publication No 2003/0147966, nanoparticles contemplated are alternatively produced using HAuCl₄ and a citrate-reducing agent, using methods known in the art. See, e.g., Marinakos et al., Adv. Mater. 11:34-37 (1999); Marinakos et al., Chem. Mater. 10: 1214-19 (1998); Enustun & Turkevich, J. Am. Chem. Soc. 85: 3317 (1963).

Nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 5 nm to about 150 nm (mean diameter), from about 30 to about 100 nm, from about 40 to about 80 um. The size of the nanoparticles used in a method varies as required by their particular use or application. The variation of size is advantageously used to optimize certain physical characteristics of the nanoparticles, for example, optical properties or the amount of surface area that can be functionalized as described herein.

### Polynucleotides

The terms “polynucleotide” and “nucleotide” or plural forms as used herein are interchangeable with modified forms as discussed herein and otherwise known in the art. In certain instances, the art uses the term “nucleobase” which embraces naturally-occurring nucleotides as well as modifications of nucleotides that can be polymerized. Thus, nucleotide or nucleobase means the naturally occurring nucleobases adenine (A), guanine (G), cytosine (C), thymine (T) and uracil (U) as well as non-naturally occurring nucleobases such as xanthine, diaminopurine, 8-oxo-N-6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Polynucleotides may also include modified nucleobases. A “modified base” is understood in the art to be one that can pair with a natural base (e.g., adenine, guanine, cytosine, uracil, and/or (hymine) and/or can pair with a non-naturally occurring base. Exemplary modified bases are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substi uted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

Nanoparticles provided that are functionalized with a polynucleotide, or modified form thereof, generally comprise a polynucleotide from about 5 nucleotides to about 100 nucleotides in length. More specifically, nanoparticles are functionalized with polynucleotide that are about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all polynucleotides intermediate in length of the sizes specifically disclosed to the extent that the polynucleotide is able to achieve the desired result. Accordingly, polynucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length are contemplated.

It is contemplated, in one embodiment, that the polynucleotide comprises one to 200 conjugation sites. In further embodiments, the polynucleotide comprises five conjugation sites. In various aspects, the polynucleotide that is functionalized on a nanoparticle comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more conjugation sites. In general, for a nucleotide, both its backbone (phosphate group) and nucleobase can be modified. Accordingly, the present disclosure contemplates that there are 2n conjugation sites, where n=length of the polynucleotide template.

### Modified Polynucleotides

Modified polynucleotides are contemplated for functionalizing nanoparticles wherein both one or more sugar and/or one or more internucleotide linkage of the nucleotide units in the polynucleotide is replaced with “non-naturally occurring” groups. In one aspect, this embodiment contemplates a peptide nucleic acid (PNA). In PNA compounds, the sugar-backbone of a polynucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

Other linkages between nucleotides and unnatural nucleotides contemplated for the disclosed polynucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et. al., Current Opinion in Structural Biology 5:343-355 (1995) and Susan M. Freier and Karl-Heinz Altmann, Nucleic Acids Research, 25:4429-4443 (1997), the disclosures of which are incorporated herein by reference.

Specific examples of polynucleotides include those containing modified backbones or non-natural internucleoside linkages. Polynucleotides having modified backbones include those that retain a phosphorus atom in the backbone and those that do not have a phosphorus atom in the backbone. Modified polynucleotides that do not have a phosphorus atom in their internucleoside backbone are considered to be within the meaning of “polynucleotide.”

Modified polynucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are polynucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated.

Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified polynucleotide backbones that do not include a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. In still other embodiments, polynucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In various forms, the linkage between two successive monomers in the oligo consists of 2 to 4, desirably 3, groups/atoms selected from —CH₂—, —O—, —S—, —NRH—, >C═O, >C═NRH, >C═S, Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)—, where RH is selected from hydrogen and C1-4-alkyl, and R″ is selected from C1-6-alkyl and phenyl. Illustrative examples of such linkages are —CH₂—CH₂—CH₂—, —CH₂—CO—CH₂—, —CH₂—CHOH—CH₂—, —O—CH2O—O, —O—CH2—CH2—, —O—CH₂—CH=(including R5 when used as a linkage to a succeeding monomer), —CH₂—CH₂—O—, —NRH—CH₂—CH₂—, —CH₂—CH₂—NRH—, —CH₂—NRH—CH₂—, —O—CH₂—CH₂—NRH—, —NRH—CO—O—, —NRH—CO—NRH—, —NRH—CS—NRH—, —NRH—C(═NRH)—NRH—, —NRH—CO—CH₂NRH—O—CO—O—, —O—CO—CH₂—O—, —O—CH₂—CO—O—, —CH₂—CO—NRH—, —O—CO—NRH—, —NRH—CO—CH₂—, —O—CH₂—CO—NRH—, —O—CH₂—CH₂—NRH—, —CH═N—O—, —CH₂NRH—, —CH₂—O—N═(including R5 when used as a linkage to a succeeding monomer), —CH₂—O—NRH—, —CO—NRH—CH₂—, —CH₂—NRH—O—, —CH₂—NRH—CO—, —O—NRH—CH₂—, —O—NRH, —O—CH₂—S—, —S—CH₂—O—, —CH₂—CH₂—S—, —O—CH₂CH₂—S—, —S—CH₂—CH₂═(including R5 when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NRH—, —NRH—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(O CH₂CH₃)—O—, —O—PO(O CH₂CH₂S—R)—O—, —O—PO(BH₃)—O—, —O—PO(NHRN)—O—, —O—P(O)₂—NRH H—, —NRH—P(O)₂—O—, —O—P(O,NRH)—O—, —CH₂—P(O)₂—O—, —O—P(O)₂—CH₂—, and —O—Si(R″)₂—O—; among which —CH₂—CO—NRH—, —CH₂—NRH—O—, —S—CH₂—O—, —O—P(O)₂—O—O—P(—O,S)—O—, —O—P(S)₂—O—, —NRH P(O)₂—O—, —O—P(O,NRH)—O—, —O—PO(R″)—O—, —O—PO(CH₃)—O—, and —O—PO(NHRN)—O—, where RH is selected form hydrogen and C1-4-alkyl, and R″ is selected from C1-6-alkyl and phenyl, are contemplated. Further illustrative examples are given in Mesmaeker et. al., 1995, Current Opinion in Structural Biology, 5: 343-355 and Susan M. Freier and Karl-Heinz Altmann, 1997, Nucleic Acids Research, vol 25: pp 4429-4443.

Still other modified forms of polynucleotides are described in detail in U.S. Patent Application No. 20040219565, the disclosure of which is incorporated by reference herein in its entirety.

Modified polynucleotides may also contain one or more substituted sugar moieties. In certain aspects, polynucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂),ONRCH₂)ₙCH₃]₂, where n and m are from 1 to about 10. Other polynucleotides comprise one of the following at the 2′ position: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a polynucleotide, or a group for improving the pharmacodynamic properties of a polynucleotide, and other substituents having similar properties. In one aspect, a modification includes 2′-methoxyethoxy (2′-O—CH₂CH₂OCH₃, also known as 2′-O-(2-methoxyethyl) or 2′-M0E) (Martin et al., 1995, Holy. Chim. Acta, 78: 486-504) i.e., an alkoxyalkoxy group. Other modifications include 2′-dimethylaminooxyethoxy, i.e., a O(CH₂)₂ON(CH₃)₂ group, also known as 2′-DMA0E, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the polynucleotide, for example, at the 3′ position of the sugar on the 3′ terminal nucleotide or in 2′-5′ linked polynucleotides and the 5′ position of 5′ terminal nucleotide. Polynucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects a methylene (—CH₂—)ₙ group bridging the 2′ oxygen atom and the 4′ carbon atom wherein n is 1 or 2. LNAs and preparation thereof are described in WO 98/39352 and WO 99/14226, the disclosures of which are incorporated herein by reference.

### Methods of Attaching Polynucleotides

Polynucleotides contemplated for use in the methods include those bound to the nanoparticle through any means. Regardless of the means by which the polynucleotide is attached to the nanoparticle, attachment in various aspects is effected through a 5′ linkage, a 3′ linkage, some type of internal linkage, or any combination of these attachments.

In one aspect, the nanoparticles, the polynucleotides or both are functionalized in order to attach the polynucleotides to the nanoparticles. Methods to functionalize nanoparticles and polynucleotides are known in the art. For instance, polynucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex., pages 109-121 (1995). See also, Mucic et al. [Chem. Commun. 555-557 (1996)] which describes a method of attaching 3′ thiol DNA to flat gold surfaces. The alkanethio]method can also be used to attach polynucleotides to other metal, semiconductor and magnetic colloids and to the other types of nanoparticles described herein. Other functional groups for attaching polynucleotides to solid surfaces include phosphorothioate groups (see, for example, U.S. Pat. No. 5,472,881 for the binding of polynucleotide-phosphorothioates to gold surfaces), substituted alkylsiloxanes [(see, for example, Burwell, Chemical Technology, 4, 370-377 (1974) and Matteucci and Canithers, J. Am. Chem. Soc., 103, 3185-3191 (1981) for binding of polynucleotides to silica and glass surfaces, and Grabar et al., [Anal. Chem., 67, 735-743] for binding of aminoalkylsiloxanes and for similar binding of mercaptoaklylsiloxanes]. Polynucleotides with a 5′ thionucleoside or a 3′ thionucleoside may also be used for attaching polynucleotides to solid surfaces. The following references describe other methods which may be employed to attached polynucleotides to nanoparticles: Nuzzo et al., J. Am. Chem. Soc., 109, 2358 (1987) (disu)fides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

U.S. patent application Ser. Nos. 09/760,500 and 09/820,279 and international application nos. PCT/US01/01190 and PCT/US01/10071 describe polynucleotides functionalized with a cyclic disulfide. The cyclic disulfides in certain aspects have 5 or 6 atoms in their rings, including the two sulfur atoms. Suitable cyclic disulfides are available commercially or are synthesized by known procedures. Functionalization with the reduced forms of the cyclic disulfides is also contemplated. Functionalization with triple cyclic disulfide anchoring groups is described in PCT/US2008/63441, incorporated herein by reference in its entirety.

In certain aspects wherein cyclic disulfide functionalization is utilized, polynucleotides are attached to a nanoparticle through one or more linkers. In one embodiment, the linker comprises a hydrocarbon moiety attached to acyclic disulfide. Suitable hydrocarbons are available commercially, and are attached to the cyclic disulfides. The hydrocarbon moiety is, in one aspect, a steroid residue. Polynucleotide-nanoparticle compositions prepared using linkers comprising a steroid residue attached to a cyclic disulfide are more stable compared to compositions prepared using alkanethiols or acyclic disulfides as the linker, and in certain instances, the polynucleotide-nanoparticle compositions have been found to be 300 times more stable. In certain embodiments the two sulfur atoms of the cyclic disulfide are close enough together so that both of the sulfur atoms attach simultaneously to the nanoparticle. In other aspects, the two sulfur atoms are adjacent each other. In aspects where utilized, the hydrocarbon moiety is large enough to present a hydrophobic surface screening the surfaces of the nanoparticle.

In other aspects, a method for attaching polynucleotides onto a surface is based on an aging process described in U.S. application Ser. No. 09/344,667, filed Jun. 25, 1999; Ser. No. 09/603,830, filed Jun. 26, 2000; Ser. No. 09/760,500, filed Jan. 12, 2001; Ser. No. 09/820,279, filed Mar. 28, 2001; Ser. No. 09/927,777, filed Aug. 10, 2001; and in International application nos. PCT/US97/12783, filed Jul. 21, 1997; PCT/US00/17507, filed Jun. 26, 2000; PCT/US01/01190, filed Jan. 12, 2001; PCT/US01/10071, filed Mar. 28, 2001, the disclosures which are incorporated by reference in their entirety. The aging process provides nanoparticle-polynucleotide compositions with enhanced stability and selectivity. The process comprises providing polynucleotides, in one aspect, having covalently bound thereto a moiety comprising a functional group which can bind to the nanoparticles. The moieties and functional groups are those that allow for binding (i.e., by chemisorption or covalent bonding) of the polynucleotides to nanoparticles. For example, polynucleotides having an alkanethiol, an alkanedisulfide or a cyclic disulfide covalently bound to their 5′ or 3′ ends bind the polynucleotides to a variety of nanoparticles, including gold nanoparticles.

Compositions produced by use of the “aging” step have been found to be considerably more stable than those produced without the “aging” step. Increased density of the polynucleotides on the surfaces of the nanoparticles is achieved by the “aging” step. The surface density achieved by the “aging” step will depend on the size and type of nanoparticles and on the length, sequence and concentration of the polynucleotides. A surface density adequate to make the nanoparticles stable and the conditions necessary to obtain it for a desired combination of nanoparticles and polynucleotides can be determined empirically. Generally, a surface density of at least 2 picomoles/cm² will be adequate to provide stable nanoparticle-polynucleotide compositions. Regardless, various polynucleotide densities are contemplated as disclosed herein.

An “aging” step is incorporated into production of functionalized nanoparticles following an initial binding or polynucleotides to a nanoparticle. In brief, the polynucleotides are contacted with the nanoparticles in water for a time sufficient to allow at least some of the polynucleotides to bind to the nanoparticles by means of the functional groups. Such times can be determined empirically. In one aspect, a time of about 12-24 hours is contemplated. Other suitable conditions for binding of the polynucleotides can also be determined empirically. For example, a concentration of about 10-20 nM nanoparticles and incubation at room temperature is contemplated.

Next, at least one salt is added to the water to form a salt solution. The salt is any water-soluble salt, including, for example and without limitation, sodium chloride, magnesium chloride, potassium chloride, ammonium chloride, sodium acetate, ammonium acetate, a combination of two or more of these salts, or one of these salts in phosphate buffer. The salt is added as a concentrated solution, or in the alternative as a solid. In various embodiments, the salt is added all at one time or the salt is added gradually over time. By “gradually over time” is meant that the salt is added in at least two portions at intervals spaced apart by a period of time. Suitable time intervals can be determined empirically.

The ionic strength of the salt solution must be sufficient to overcome at least partially the electrostatic repulsion of the polynucleotides from each other and, either the electrostatic attraction of the negatively-charged polynucleotides for positively-charged nanoparticles, or the electrostatic repulsion of the negatively-charged polynucleotides from negatively-charged nanoparticles. Gradually reducing the electrostatic attraction and repulsion by adding the salt gradually over time gives the highest surface density of polynucleotides on the nanoparticles. Suitable ionic strengths can be determined empirically for each salt or combination of salts. In one aspect, a final concentration of sodium chloride of from about 0.01 M to about 1.0 M in phosphate buffer is utilized, with the concentration of sodium chloride being increased gradually over time. In another aspect, a final concentration of sodium chloride of from about 0.01 M to about 0.5 M, or about 0.1 M to about 0.3 M is utilized, with the concentration of sodium chloride being increased gradually over time.

After adding the salt, the polynucleotides and nanoparticles are incubated in the salt solution for a period of time to allow additional polynucleotides to bind to the nanoparticles to produce the stable nanoparticle-polynucleotide compositions. An increased surface density of the polynucleotides on the nanoparticles stabilizes the compositions, as has been described herein. The time of this incubation can be determined empirically. By way of example, in one aspect a total incubation time of about 24-48, wherein the salt concentration is increased gradually over this total time, is contemplated. This second period of incubation in the salt solution is referred to herein as the “aging” step. Other suitable conditions for this “aging” step can also be determined empirically. By way of example, an aging step is carried out with incubation at room temperature and pH 7.0.

The compositions produced by use of the “aging” are in general more stable than those produced without the “aging” step. As noted above, this increased stability is due to the increased density of the polynucleotides on the surfaces of the nanoparticles which is achieved by the “aging” step. The surface density achieved by the “aging” step will depend on the size and type of nanoparticles and on the length, sequence and concentration of the polynucleotides.

As used herein, “stable” means that, for a period of at least six months after the compositions are made, a majority of the polynucleotides remain attached to the nanoparticles and the polynucleotides are able to hybridize with nucleic acid and polynucleotide targets under standard conditions encountered in methods of detecting nucleic acid and methods of nanofabrication.

### Surface Density

Nanoparticles provided herein have a packing density of the polynucleotides on the surface of the nanoparticle that is, in various aspects, sufficient to result in cooperative behavior between nanoparticles and between polynucleotide strands on a single nanoparticle. In another aspect, the cooperative behavior between the nanoparticles increases the resistance of the polynucleotide to nuclease degradation. In yet another aspect, the uptake of nanoparticles by a cell is influenced by the density of polynucleotides associated with the nanoparticle. As described in PCT/US2008/65366, incorporated herein by reference in its entirety, a higher density of polynucleotides on the surface of a nanoparticle is associated with an increased uptake of nanoparticles by a cell.

A surface density adequate to make the nanoparticles stable and the conditions necessary to obtain it for a desired combination of nanoparticles and polynucleotides can be determined empirically. Generally, a surface density of at least 2 pmoles/cm² will be adequate to provide stable nanoparticle-polynucleotide compositions. In some aspects, the surface density is at least 15 pmoles/cm². Methods are also provided wherein the polynucleotide is bound to the nanoparticle at a surface density of at least 2 μmol/cm², at least 3 μmol/cm², at least 4 μmol/cm², at least 5 μmol/cm², at least 6 μmol/cm², at least 7 μmol/cm², at least 8 μmol/cm², at least 9 μmol/cm², at least 10 μmol/cm², at least about 15 μmol/cm², at least about 20 μmol/cm², at least about 25 μmol/cm², at least about 30 μmol/cm², at least about 35 μmol/cm², at least about 40 μmol/cm², at least about 45 μmol/cm², at least about 50 μmol/cm², at least about 55 μmol/cm least about 60 μmol/cm², at least about 65 μmol/cm², at least about 70 μmol/cm², at least about 75 μmol/cm², at least about 80 μmol/cm², at least about 85 μmol/cm², at least about 90 μmol/cm² at least about 95 μmol/cm², at least about 100 μmol/cm², at least about 125 μmol/cm², at least about 150 μmol/cm², at least about 175 μmol/cm², at least about 200 μmol/cm², at least about 250 μmol/cm², at least about 300 pmol/cm², at least about 350 μmol/cm², at least about 400 μmol/cm², at least about 450 μmol/cm², at least about 500 μmol/cm², at least about 550 μmol/cm², at least about 600 μmol/cm², at least about 650 μmol/cm², at least about 700 μmol/cm², at least about 750 μmol/cm², at least about 800 μmol/cm², at least about 850 μmol/cm², at least about 900 μmol/cm², at least about 950 μmol/cm², at least about 1000 μmol/cm² or more.

Density of polynucleotides on the surface of a nanoparticle has been shown to modulate specific polypeptide interactions with the polynucleotide on the surface and/or with the nanoparticle itself Under various conditions, some polypeptides may be prohibited from interacting with polynucleotides associated with a nanoparticle based on steric hindrance caused by the density of polynucleotides. In aspects where interaction of polynucleotides with polypeptides that are otherwise precluded by steric hindrance is desirable, the density of polynucleotides on the nanoparticle surface is decreased to allow the polypeptide to interact with the polynucleotide.

Polynucleotide surface density has also been shown to modulate stability of the polynucleotide associated with the nanoparticle. In one embodiment, an RNA polynucleotide associated with a nanoparticle is provided wherein the RNA polynucleotide has a half-life that is at least substantially the same as the half-life of an identical RNA polynucleotide that is not associated with a nanoparticle. In other embodiments, the RNA polynucleotide associated with the nanoparticle has a half-life that is about 5% greater, about 10% greater, about 20% greater, about 30% greater, about 40% greater, about 50% greater, about 60% greater, about 70% greater, about 80% greater, about 90% greater, about 2-fold greater, about 3-fold greater, about 4-fold greater, about 5-fold greater, about 6-fold greater, about 7-fold greater, about 8-fold greater, about 9-fold greater, about 10-fold greater, about 20-fold greater, about 30-fold greater, about 40-fold greater, about 50-fold greater, about 60-fold greater, about 70-fold greater, about 80-fold greater, about 90-fold greater, about 100-fold greater, about 200-fold greater, about 300-fold greater, about 400-fold greater, about 500-fold greater, about 600-fold greater, about 700-fold greater, about 800-fold greater, about 900-fold greater, about 1000-fold greater, about 5000-fold greater, about 10,000-fold greater, about 50,000-fold greater, about 100,000-fold greater, about 200,000-fold greater, about 300,000-fold greater, about 400,000-fold greater, about 500,000-fold greater, about 600,000-fold greater, about 700,000-fold greater, about 800,000-fold greater, about 900,000-fold greater, about 1,000,000-fold greater or more than the half-life of an identical RNA polynucleotide that is not associated with a nanoparticle.

Nanoparticles of larger diameter are, in some aspects, contemplated to be functionalized with a greater number of polynucleotides [Hurst et al., Analytical Chemistry 78(24): 8313-8318 (2006)]. In some aspects, therefore, the number of polynucleotides functionalized on a nanoparticle is from about 10 to about 25,000 polynucleotides per nanoparticle. In further aspects, the number of polynucleotides functionalized on a nanoparticle is from about 50 to about 10,000 polynucleotides per nanoparticle, and in still further aspects the number of polynucleotides functionalized on a nanoparticle is from about 200 to about 5,000 polynucleotides per nanoparticle. In various aspects, the number of polynucleotides functionalized on a nanoparticle is about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500, about 505, about 510, about 515, about 520, about 525, about 530, about 535, about 540, about 545, about 550, about 555, about 560, about 565, about 570, about 575, about 580, about 585, about 590, about 595, about 600, about 605, about 610, about 615, about 620, about 625, about 630, about 635, about 640, about 645, about 650, about 655, about 660, about 665, about 670, about 675, about 680, about 685, about 690, about 695, about 700, about 705, about 710, about 715, about 720, about 725, about 730, about 735, about 740, about 745, about 750, about 755, about 760, about 765, about 770, about 775, about 780, about 785, about 790, about 795, about 800, about 805, about 810, about 815, about 820, about 825, about 830, about 835, about 840, about 845, about 850, about 855, about 860, about 865, about 870, about 875, about 880, about 885, about 890, about 895, about 900, about 905, about 910, about 915, about 920, about 925, about 930, about 935, about 940, about 945, about 950, about 955, about 960, about 965, about 970, about 975, about 980, about 985, about 990, about 995, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5200, about 5300, about 5400, about 5500, about 5600, about 5700, about 5800, about 5900, about 6000, about 6100, about 6200, about 6300, about 6400, about 6500, about 6600, about 6700, about 6800, about 6900, about 7000, about 7100, about 7200, about 7300, about 7400, about 7500, about 7600, about 7700, about 7800, about 7900, about 8000, about 8100, about 8200, about 8300, about 8400, about 8500, about 8600, about 8700, about 8800, about 8900, about 9000, about 9100, about 9200, about 9300, about 9400, about 9500, about 9600, about 9700, about 9800, about 9900, about 10000, about 10100, about 10200, about 10300, about 10400, about 10500, about 10600, about 10700, about 10800, about 10900, about 11000, about 11100, about 11200, about 11300, about 11400, about 11500, about 11600, about 11700, about 11800, about 11900, about 12000, about 12100, about 12200, about 12300, about 12400, about 12500, about 12600, about 12700, about 12800, about 12900, about 13000, about 13100, about 13200, about 13300, about 13400, about 13500, about 13600, about 13700, about 13800, about 13900, about 14000, about 14100, about 14200, about 14300, about 14400, about 14500, about 14600, about 14700, about 14800, about 14900, about 15000, about 15100, about 15200, about 15300, about 15400, about 15500, about 15600, about 15700, about 15800, about 15900, about 16000, about 16100, about 16200, about 16300, about 16400, about 16500, about 16600, about 16700, about 16800, about 16900, about 17000, about 17100, about 17200, about 17300, about 17400, about 17500, about 17600, about 17700, about 17800, about 17900, about 18000, about 18100, about 18200, about 18300, about 18400, about 18500, about 18600, about 18700, about 18800, about 18900, about 19000, about 19100, about 19200, about 19300, about 19400, about 19500, about 19600, about 19700, about 19800, about 19900, about 20000, about 20100, about 20200, about 20300, about 20400, about 20500, about 20600, about 20700, about 20800, about 20900, about 21000, about 21100, about 21200, about 21300, about 21400, about 21500, about 21600, about 21700, about 21800, about 21900, about 22000, about 22100, about 22200, about 22300, about 22400, about 22500, about 22600, about 22700, about 22800, about 22900, about 23000, about 23100, about 23200, about 23300, about 23400, about 23500, about 23600, about 23700, about 23800, about 23900, about 24000, about 24100, about 24200, about 24300, about 24400, about 24500, about 24600, about 24700, about 24800, about 24900, about 25000 or more per nanoparticle.

### Polynucleotide Features

In some aspects, the polynucleotide that is functionalized to the nanoparticle allows for efficient uptake of the PN-NP. In various aspects, the polynucleotide comprises a nucleotide sequence that allows increased uptake efficiency of the PN-NP. As used herein, “efficiency” refers to the number or rate of uptake of nanoparticles in/by a cell. Because the process of nanoparticles entering and exiting a cell is a dynamic one, efficiency can be increased by taking up more nanoparticles or by retaining those nanoparticles that enter the cell for a longer period of time. Similarly, efficiency can be decreased by taking up fewer nanoparticles or by retaining those nanoparticles that enter the cell for a shorter period of time.

The nucleotide sequence can be any nucleotide sequence that is desired may be selected for, in various aspects, increasing or decreasing cellular uptake of a PN-NP or gene regulation. The nucleotide sequence, in some aspects, comprises a homopolymeric sequence which affects the efficiency with which the nanoparticle to which the polynucleotide is attached is taken up by a cell. Accordingly, the homopolymeric sequence increases or decreases the efficiency. It is also contemplated that, in various aspects, the nucleotide sequence is a combination of nucleobases, such that it is not strictly a homopolymeric sequence. For example and without limitation, in various aspects, the nucleotide sequence comprises alternating thymidine and uridine residues, two thymidines followed by two uridines or any combination that affects increased uptake is contemplated by the disclosure. In some aspects, the nucleotide sequence affecting uptake efficiency is included as a domain in a polynucleotide comprising additional sequence. This “domain” would serve to function as the feature affecting uptake efficiency, while the additional nucleotide sequence would serve to function, for example and without limitation, to regulate gene expression. In various aspects, the domain in the polynucleotide can be in either a proximal, distal, or center location relative to the nanoparticle. It is also contemplated that a polynucleotide comprises more than one domain.

The homopolymeric sequence, in some embodiments, increases the efficiency of uptake of the polynucleotide-functionalized nanoparticle by a cell. In some aspects, the homopolymeric sequence comprises a sequence of thymidine residues (polyT) or uridine residues (polyU). In further aspects, the polyT or polyU sequence comprises two thymidines or uridines. In various aspects, the polyT or polyU sequence comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 125, about 150, about 175, about 200, about 250, about 300, about 350, about 400, about 450, about 500 or more thymidine or uridine residues.

In some embodiments, it is contemplated that a nanoparticle functionalized with a polynucleotide comprising a homopolymeric sequence is taken up by a cell with greater efficiency than a nanoparticle functionalized with the same polynucleotide but lacking the homopolymeric sequence. In some aspects, a nanoparticle functionalized with a polynucleotide and a homopolymeric sequence is taken up by a cell 1% more efficiently than a nanoparticle functionalized with the same polynucleotide but lacking the homopolymeric sequence. In various aspects, a nanoparticle functionalized with a polynucleotide and a homopolymeric sequence is taken up by a cell 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 100-fold or higher, more efficiently than a nanoparticle functionalized with the same polynucleotide but lacking the homopolymeric sequence.

The methods of the disclosure also provide, in certain aspects, one or more polynucleotides that are functionalized to the nanoparticle that do not comprise a conjugation site while one or more polynucleotides on the same nanoparticle do comprise a conjugation site. In these aspects, it is contemplated that the composition comprises a nanoparticle to which a plurality of polynucleotides are attached. In some aspects, the plurality of polynucleotides comprises at least one polynucleotide to which contrast agents are associated through one or more conjugation sites, as well as at least one polynucleotide that has gene regulatory activity as described herein.

Accordingly, in some embodiments, it is contemplated that one or more polynucleotides functionalized to the nanoparticle is not conjugated to a contrast agent while one or more polynucleotides on the same nanoparticle are conjugated to a contrast agent. In some aspects, the PN-NP is functionalized with DNA. In some embodiments, the DNA is double stranded, and in further embodiments the DNA is single stranded. In further aspects, the PN-NP is functionalized with RNA, and in still further aspects the PN-NP is functionalized with double stranded RNA agents known as small interfering RNA (siRNA). The term “RNA” includes duplexes of two separate strands, as well as single stranded structures. Single stranded RNA also includes RNA with secondary structure. In one aspect, RNA having a hairpin loop in contemplated.

Polynucleotides that are contemplated for use in gene regulation and functionalized to a nanoparticle have complementarity to (i.e., are able to hybridize with) a portion of a target RNA (generally messenger RNA (mRNA)). The polynucleotide can further comprise a conjugation site to which a contrast agent can bind.

“Hybridization” means an interaction between two or three strands of nucleic acids by hydrogen bonds in accordance with the rules of Watson-Crick complementarity, Hoogstein binding, or other sequence-specific binding known in the art. Hybridization can be performed under different stringency conditions known in the art.

Generally, such complementarity is 100%, but can be less if desired, such as about 20%, about 25%, about 30%, about 35%, about 40%, about 45%, about 50%, about 55%, about 60%, about 70%̂ 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. For example, 19 bases out of 21 bases may be base-paired. Thus, it will be understood that a polynucleotide used in the methods need not be 100% complementary to a desired target nucleic acid to be specifically hybridizable. Moreover, polynucleotides may hybridize to each other over one or more segments such that intervening or adjacent segments are not involved in the hybridization event (e.g., a loop structure or hairpin s(ructure). Percent complementarity between any given polynucleotide can be determined routinely using BLAST programs (Basic Local Alignment Search Tools) and PowerBLAST programs known in the art (Altschul et al., 1990, J. Mol. Biol., 215: 403-4)0; Zhang and Madden, 1997, Genome Res., 7: 649-656).

In some aspects, where selection between various allelic variants is desired, 100% complementarity to the target gene is required in order to effectively discern the target sequence from the other allelic sequence. When selecting between allelic targets, choice of length is also an important factor because it is the other factor involved in the percent complementary and the ability to differentiate between allelic differences.

### Target Polynucleotide Sequences and Hybridization

In some aspects, the disclosure provides methods of targeting specific polynucleotide. Any type of polynucleotide may be targeted, and the methods may be used, e.g., for therapeutic modulation of gene expression (See, e.g., PCT/US2006/022325, the disclosure of which is incorporated herein by reference). Examples of polynucleotides that can be targeted by the methods of the invention include but are not limited to genes (e.g., a gene associated with a particular disease), viral RNA, mRNA, RNA, or single-stranded nucleic acids.

The target nucleic acid may be in cells or biological fluids, as also known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995).

The terms “start codon region” and “translation initiation codon region” refer to a portion of a mRNA or gene that encompasses contiguous nucleotides in either direction (i.e., 5′ or 3′) from a translation initiation codon. Similarly, the terms “stop codon region” and “translation termination codon region” refer to a portion of such a mRNA or gene that encompasses contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the polynucleotides on the functionalized nanoparticles.

Other target regions include the 5′ untranslated region (5′UTR), the portion of an mRNA in the 5′ direction from the translation initiation codon, including nucleotides between the 5′ cap site and the translation initiation codon of a mRNA (or corresponding nucleotides on the gene), and the 3′ untranslated region (3′UTR), the portion of a mRNA in the 3′ direction from the translation termination codon, including nucleotides between the translation termination codon and 3′ end of a mRNA (or corresponding nucleotides on the gene). The 5′ cap site of a mRNA comprises an N7-methylated guanosine residue joined to the 5′-most residue of the mRNA via a 5′-5′ triphosphate linkage. The 5′ cap region of a mRNA is considered to include the 5′ cap structure itself as well as the first 50 nucleotides adjacent to the cap site.

For prokaryotic target nucleic acid, in various aspects, the nucleic acid is RNA transcribed from genomic DNA. For eukaryotic target nucleic acid, the nucleic acid is an animal nucleic acid, a fungal nucleic acid, including yeast nucleic acid. As above, the target nucleic acid is a RNA transcribed from a genomic DNA sequence. In certain aspects, the target nucleic acid is a mitochondrial nucleic acid. For viral target nucleic acid, the nucleic acid is viral genomic RNA, or RNA transcribed from viral genomic DNA.

In some embodiments of the disclosure, a target polynucleotide sequence is a microRNA. MicroRNAs (miRNAs) are 20-22 nucleotide (nt) molecules generated from longer 70-nt RNAs that include an imperfectly complementary hairpin segment [Jackson et al., Sri STKE 367: rel (2007); Mendell, Cell Cycle 4: 1179-1184 (2005)]. The longer precursor molecules are cleaved by a group of proteins (Drosha and DCGR8) in the nucleus into smaller RNAs called pre-miRNA. Pre-miRNAs are then exported into the cytoplasm by exportin [Vinuani et al., J Vasc Intery Radiol 19: 931-936 (2008)] proteins. The pre-miRNA in the cytoplasm is then cleaved into mature RNA by a complex of proteins called RNAi silencing complex or RISC. The resulting molecule has 19-bp double stranded RNA and 2 nt 3′ overhangs on both strands. One of the two strands is then expelled from the complex and is degraded. The resulting single strand RNA-protein complex can then inhibit translation (either by repressing the actively translating ribosomes or by inhibiting initiation of translation) or enhance degradation of the mRNA it is attached to. There is, of course, a high degree of selectivity to this process, as the miRNA only binds to areas that are of high match to its sequence [Zamore et al., Science 309: 1519-1524 (2005)]. In one aspect, the target polynucleotide is microRNA-210.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of an polynucleotide-functionalized nanoparticle. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of nanoparticle and a specific polynucleotide.

### Contrast Agents

Disclosed herein are methods and compositions comprising a nanoparticle functionalized with a polynucleotide, wherein the polynucleotide is conjugated to a contrast agent through a conjugation site. As used herein, a “contrast agent” is a compound or other substance introduced into a cell in order to create a difference in the apparent density of various organs and tissues, making it easier to see the delineate adjacent body tissues and organs.

As described in U.S. Patent Application Number 2010/0183504, the disclosure of which is incorporated herein in its entirety, the performance of a contrast agent in solution is measured by its relaxivity, defined as 1/Tᵢ˜rᵢ*[C], i=1,2, where rᵢ is the relaxivity and [C] the concentration of the contrast agent. The rule is that the higher its relaxivity, the more sensitive the contrast agent. T₁-contrast agents are agents that affect mostly the longitudinal relaxation time. In various aspect, these contrast agent are made of chelated lanthanide ions and reach relaxivities of 5-30 mM⁻¹ s⁻¹. Higher relaxivities are obtained with T₂-contrast agents, i.e. agents that affect mainly the transversal relaxation time, the most prominent of which are small superparamagnetic iron oxide nanoparticles (SPIO) [Wang et al., Nano Lett. 8(11): 3761-5 (2008)]. These particles are under heavy investigation for studying stem cells or the spatial distribution of immuno-competent cells in tumors over time. SPIO have sizes typically ranging from approximately 30-50 nm in diameter. They contain thousands of iron atoms and reach relaxivities of up to 200 mM⁻¹ s⁻¹.

Methods provided by the disclosure include those wherein relaxivity of the contrast agent in association with a nanoparticle is increased relative to the relaxivity of the contrast agent in the absence of being associated with a nanoparticle. In some aspects, the increase is about 1-fold to about 20-fold. In further aspects, the increase is about 2-fold fold to about 10-fold, and in yet further aspects the increase is about 3-fold.

The increase in relaxivity of the contrast agent in association with a nanoparticle is, in various embodiments, about 1-fold, about 1.5-fold, about 2-fold, about 2.5-fold, about 3-fold, about 3.5-fold, about 4-fold, about 4.5-fold, about 5-fold, about 5.5-fold, about 6-fold, about 6.5-fold, about 7-fold, about 7.5-fold, about 8-fold, about 8.5-fold, about 9-fold, about 9.5-fold, about 10-fold, about 10.5-fold, about 11-fold, about 11.5-fold, about 12-fold, about 12.5-fold, about 13-fold, about 13.5-fold, about 14-fold, about 14.5-fold, about 15-fold, about 15.5-fold, about 16-fold, about 16.5-fold, about 17-fold, about 17.5-fold, about 18-fold, about 18.5-fold, about 19-fold, about 19.5-fold, about 20-fold or higher relative to the relaxivity of the contrast agent in the absence of being associated with a nanoparticle.

In some embodiments, the contrast agent is selected from the group consisting of gadolinium, xenon, iron oxide, a manganese chelate (Mn-DPDP) and copper. Thus, in some embodiments the contrast agent is a paramagnetic compound, and in some aspects, the paramagnetic compound is gadolinium.

The present disclosure also contemplates contrast agents that are useful for positron emission tomography (PET) scanning. In some aspects, the PET contrast agent is a radionuclide. In certain embodiments the contrast agent comprises a PET contrast agent comprising a label selected from the group consisting of ¹¹C, ¹³N, ¹⁸F, ⁶⁴Cu, ⁶⁸Ge, ^{99m}Tc and ⁸²Ru. In particular embodiments the contrast agent is a PET contrast agent selected from the group consisting of [¹¹C]choline, [¹⁸F] fluorodeoxyglucose(FDG), [¹¹C]methionine, [¹¹C]choline, [¹¹C]acetate, [¹⁸F]fluorocholine, ⁶⁴Cu chelates, ^{99m}Tc chelates, and [¹⁸F]polyethyleneglycol stilbenes.

The disclosure also provides methods wherein a PET contrast agent is introduced into a polynucleotide during the polynucleotide synthesis process or is conjugated to a nucleotide following polynucleotide synthesis. For example and without limitation, nucleotides can be synthesized in which one of the phosphorus atoms is replaced with ³²P or ³³P, one of the oxygen atoms in the phosphate group is replaced with ³⁵S, or one or more of the hydrogen atoms is replaced with ³H. A functional group containing a radionuclide can also be conjugated to a nucleotide through conjugation sites.

The MRI contrast agents can include, but are not limited to positive contrast agents and/or negative contrast agents. Positive contrast agents cause a reduction in the T₁ relaxation time (increased signal intensity on T₁ weighted images). They (appearing bright on MRI) are typically small molecular weight compounds containing as their active element Gadolinium, Manganese, or Iron. All of these elements have unpaired electron spins in their outer shells and long relaxivities. A special group of negative contrast agents (appearing dark on MRI) include perfluorocarbons (perfluorochemicals), because their presence excludes the hydrogen atoms responsible for the signal in MR imaging.

The composition of the disclosure, in various aspects, is contemplated to comprise a nanoparticle that comprises about 50 to about 2.5×10⁶ contrast agents. In some embodiments, the nanoparticle comprises about 500 to about 1×10⁶ contrast agents. In various aspects, the disclosure contemplates that the compositions described herein comprise a nanoparticle that comprises about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 260, about 270, about 280, about 290, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5200, about 5300, about 5400, about 5500, about 5600, about 5700, about 5800, about 5900, about 6000, about 6100, about 6200, about 6300, about 6400, about 6500, about 6600, about 6700, about 6800, about 6900, about 7000, about 7100, about 7200, about 7300, about 7400, about 7500, about 7600, about 7700, about 7800, about 7900, about 8000, about 8100, about 8200, about 8300, about 8400, about 8500, about 8600, about 8700, about 8800, about 8900, about 9000, about 9100, about 9200, about 9300, about 9400, about 9500, about 9600, about 9700, about 9800, about 9900, about 10000, about 10500, about 11000, about 11500, about 12000, about 12500, about 13000, about 13500, about 14000, about 14500, about 15000, about 15500, about 16000, about 16500, about 17000, about 17500, about 18000, about 18500, about 19000, about 19500, about 20000, about 20500, about 21000, about 21500, about 22000, about 22500, about 23000, about 23500, about 24000, about 24500, about 25000, about 25500, about 26000, about 26500, about 27000, about 27500, about 28000, about 28500, about 29000, about 29500, about 30000, about 30500, about 31000, about 31500, about 32000, about 32500, about 33000, about 33500, about 34000, about 34500, about 35000, about 35500, about 36000, about 36500, about 37000, about 37500, about 38000, about 38500, about 39000, about 39500, about 40000, about 40500, about 41000, about 41500, about 42000, about 42500, about 43000, about 43500, about 44000, about 44500, about 45000, about 45500, about 46000, about 46500, about 47000, about 47500, about 48000, about 48500, about 49000, about 49500, about 50000, about 15000, about 20000, about 25000, about 30000, about 35000, about 40000, about 45000, about 50000, about 55000, about 60000, about 65000, about 70000, about 75000, about 80000, about 85000, about 90000, about 95000, about 100000, about 105000, about 110000, about 115000, about 120000, about 125000, about 130000, about 135000, about 140000, about 145000, about 150000, about 155000, about 160000, about 165000, about 170000, about 175000, about 180000, about 185000, about 190000, about 195000, about 200000, about 205000, about 210000, about 215000, about 220000, about 225000, about 230000, about 235000, about 240000, about 245000, about 250000, about 255000, about 260000, about 265000, about 270000, about 275000, about 280000, about 285000, about 290000, about 295000, about 300000, about 305000, about 310000, about 315000, about 320000, about 325000, about 330000, about 335000, about 340000, about 345000, about 350000, about 355000, about 360000, about 365000, about 370000, about 375000, about 380000, about 385000, about 390000, about 395000, about 400000, about 405000, about 410000, about 415000, about 420000, about 425000, about 430000, about 435000, about 440000, about 445000, about 450000, about 455000, about 460000, about 465000, about 470000, about 475000, about 480000, about 485000, about 490000, about 495000, about 500000, about 550000, about 600000, about 650000, about 700000, about 750000, about 800000, about 850000, about 900000, about 950000, about 1000000, about 1050000, about 1100000, about 1150000, about 1200000, about 1250000, about 1300000, about 1350000, about 1400000, about 1450000, about 1500000, about 1550000, about 1600000, about 1650000, about 1700000, about 1750000, about 1800000, about 1850000, about 1900000, about 1950000, about 2000000, about 2050000, about 2100000, about 2150000, about 2200000, about 2250000, about 2300000, about 2350000, about 2400000, about 2450000, about 2500000 or more contrast agents.

### Imaging Procedures

### Magnetic Resonance Imaging (MRI)

Magnetic resonance imaging is a method often used for in vivo visualization because of its infinite penetration depth and its anatomic resolution. MRI maps the relaxation processes of water protons in the sample, referred to as T₁ and T₂ relaxation times. One of the powers of MRI is its ability to extract image contrast, or a difference in image intensity between tissues, on the basis of variations in the local environment of mobile water. Unfortunately, as naturally-occurring molecules in cells lack useful fluorescence properties for imaging, intrinsic differences between tissues are often too small to provide distinguishable relaxation times. This is why exogenous contrast agents are often used, most notably in the form of small amounts of paramagnetic impurities. The paramagnetic materials accelerate the T₁ and T₂ relaxation processes of water protons in their surroundings.

MRI is widely used clinically because it provides high spatial resolution images, particularly through the application of contrast agents which are currently employed in approximately 35% of all clinical MRI examinations. These are typically derived from iron particles or paramagnetic, predominantly Gd, complexes. One of the clinically approved, and commonly used contrast agents are Gd-DOTA (DOTA=1,4,7,10-tetrakis(carboxymethyl)-1,4,7,10-tetraazacyclodode-cane), which shows low toxicity and patient discomfort. Clinical safety results from its low osmolality, low viscosity, low chemotoxicity, high solubility, and high in vivo stability for the macrocylic complex.

The vast majority of MRI applications depend on the bulk biodistribution of the contrast agent rather than molecular targeting methods. As a small molecule, Gd agents get into the microvasculature around tumors, which is at a much higher density than normal tissue. This increased concentration of Gd in highly vascularized tissue around tumors is the basis for the MRI contrast mechanism. Thus, compositions able to specifically enter cells, as described herein, are extremely useful for improving the ability of MRI to localize cancer.

In certain embodiments, the MRI contrast agent conjugated to a polynucleotide is iron or paramagnetic radiotracers and/or complexes, including but not limited to gadolinium, xenon, iron oxide, and copper.

### Computed Tomography (CT)

Digital geometry processing is used to generate a three-dimensional image of the inside of an object from a large series of two-dimensional X-ray images taken around a single axis of rotation [Herman, Fundamentals of computerized tomography: Image reconstruction from projection, 2nd edition, Springer, (2009)].

CT produces a volume of data which can be manipulated, through a process known as “windowing”, in order to demonstrate various bodily structures based on their ability to block the X-ray beam. Although historically the images generated were in the axial or transverse plane, orthogonal to the long axis of the body, modern scanners allow this volume of data to be reformatted in various planes or even as volumetric (3D) representations of structures.

CT scanning of the head is typically used to detect infarction, tumors, calcifications, hemorrhage and bone trauma.

Of the above, hypodense (dark) structures indicate infraction or tumors, hyperdense (bright) structures indicate calcifications and hemorrhage and bone trauma can be seen as disjunction in bone windows.

CT can be used for detecting both acute and chronic changes in the lung parenchyma, that is, the internals of the lungs. It is particularly relevant because normal two dimensional x-rays do not show such defects. A variety of different techniques are used depending on the suspected abnormality. For evaluation of chronic interstitial processes (emphysema, fibrosis, and so forth), thin sections with high spatial frequency reconstructions are used. This special technique is called High Resolution CT (HRCT). HRCT is normally done with thin section with skipped areas between the thin sections. Therefore it produces a sampling of the lung and not continuous images. Continuous images are provided in a standard CT of the chest.

For detection of airspace disease (such as pneumonia) or cancer, relatively thick sections and general purpose image reconstruction techniques may be adequate. IV contrast may also be used as it clarifies the anatomy and boundaries of the great vessels and improves assessment of the mediastinum and hilar regions for lymphadenopathy; this is particularly important for accurate assessment of cancer.

CT angiography of the chest is also becoming the primary method for detecting pulmonary embolism (PE) and aortic dissection, and requires accurately timed rapid injections of contrast (Bolus Tracking) and high-speed helical scanners. CT is the standard method of evaluating abnolinalities seen on chest X-ray and of following findings of uncertain acute significance. CT pulmonary angiogram (CTPA) is a medical diagnostic test used to diagnose pulmonary embolism (PE). It employs computed tomography to obtain an image of the pulmonary arteries. A normal CTPA scan will show the contrast filling the pulmonary vessels, looking bright white. Ideally the aorta should be empty of contrast, to reduce any partial volume artifact which may result in a false positive. Any mass filling defects, such as an embolus, will appear dark in place of the contrast, filling/blocking the space where blood should be flowing into the lungs.

With the advent of sub second rotation combined with multi-slice CT (up to 64-slice), high resolution and high speed can be obtained at the same time, allowing excellent imaging of the coronary arteries (cardiac CT angiography). Images with an even higher temporal resolution can be formedusing retrospective ECG gating. In this technique, each portion of the heart is imaged more than once while an ECG trace is recorded. The ECG is then used to correlate the CT data with their corresponding phases of cardiac contraction. Once this correlation is complete, all data that were recorded while the heart was in motion (systole) can be ignored and images can be made from the remaining data that happened to be acquired while the heart was at rest (diastole). In this way, individual frames in a cardiac CT investigation have abetter temporal resolution than the shortest tube rotation time.

CT is a sensitive method for diagnosis of abdominal diseases. It is used frequently to determine stage of cancer and to follow progress. It is also a useful test to investigate acute abdominal pain (especially of the lower quadrants, whereas ultrasound is the preferred first line investigation for right upper quadrant pain). Renal stones, appendicitis, pancreatitis, diverticulitis, abdominal aortic aneurysm, and bowel obstruction are conditions that are readily diagnosed and assessed with CT. CT is also the first line for detecting solid organ injury after trauma.

CT is often used to image complex fractures, especially ones around joints, because of its ability to reconstruct the area of interest in multiple planes. Fractures, ligamentous injuries and dislocations can easily be recognized with a 0.2 mm resolution.

### X-Ray Attenuation

X-ray photons used for medical purposes are formed by an event involving an electron, while gamma ray photons are formed from an interaction with the nucleus of an atom [Radiation Detection and Measurement 3rd Edition, Glenn F. Knoll: Chapter 1, Page 1: John Wiley & Sons; 3rd Edition edition (26 Jan. 2000)]. In general, medical radiography is done using X-rays formed in an X-ray tube. Nuclear medicine typically involves gamma rays.

The types of electromagnetic radiation of most interest to radiography are X-ray and gamma radiation. This radiation is much more energetic than the more familiar types such as radio waves and visible light. It is this relatively high energy which makes gamma rays useful in radiography but potentially hazardous to living organisms.

The radiation is produced by X-ray tubes, high energy X-ray equipment or natural radioactive elements, such as radium and radon, and artificially produced radioactive isotopes of elements, such as cobalt-60 and iridium-192. Electromagnetic radiation consists of oscillating electric and magnetic fields, but is generally depicted as a single sinusoidal wave.

Gamma rays are indirectly ionizing radiation. A gamma ray passes through matter until it undergoes an interaction with an atomic particle, usually an electron. During this interaction, energy is transferred from the gamma ray to the electron, which is a directly ionizing particle. As a result of this energy transfer, the electron is liberated from the atom and proceeds to ionize matter by colliding with other electrons along its path. Other times, the passing gamma ray interferes with the orbit of the electron, and slows it, releasing energy but not becoming dislodged. The atom is not ionised, and the gamma ray continues on, although at a lower energy. This energy released is usually heat or another, weaker photon, and causes biological harm as a radiation burn. The chain reaction caused by the initial dose of radiation can continue after exposure.

For the range of energies commonly used in radiography, the interaction between gamma rays and electrons occurs in two ways. One effect takes place where all the gamma ray's energy is transmitted to an entire atom. The gamma ray no longer exists and an electron emerges from the atom with kinetic (motion in relation to force) energy almost equal to the gamma energy. This effect is predominant at low gamma energies and is known as the photoelectric effect. The other major effect occurs when a gamma ray interacts with an atomic electron, freeing it from the atom and imparting to it only a fraction of the gamma ray's kinetic energy. A secondary gamma ray with less energy (hence lower frequency) also emerges from the interaction. This effect predominates at higher gamma energies and is known as the Compton effect.

In both of these effects the emergent electrons lose their kinetic energy by ionizing surrounding atoms. The density of ions so generated is a measure of the energy delivered to the material by the gamma rays.

The most common means of measuring the variations in a beam of radiation is by observing its effect on a photographic film. This effect is the same as that of light, and the more intense the radiation is, the more it darkens, or exposes, the film. Other methods are in use, such as the ionizing effect measured electronically, its ability to discharge an electrostatically charged plate or to cause certain chemicals to fluoresce as in fluoroscopy.

### Luminescence

A luminophore as described herein is an atom or atomic grouping in a chemical compound that manifests luminescence. There exist organic and inorganic luminophores. Luminescence is light that usually occurs at low temperatures, and is thus a form of cold body radiation. It can be caused by chemical reactions, electrical energy, subatomic motions, or stress on a crystal.

### Near-Infrared Spectroscopy

Near-infrared spectroscopy (NIRS) is a spectroscopic method that uses the near-infrared region of the electromagnetic spectrum (from about 800 nm to 2500 nm). Typical applications include pharmaceutical, medical diagnostics (including blood sugar and oximetry), food and agrochemical quality control, as well as combustion research.

Medical applications of NIRS center on the non-invasive measurement of the amount and oxygen content of hemoglobin, as well as the use of exogenous optical tracers in conjunction with flow kinetics.

NIRS can be used for non-invasive assessment of brain function through the intact skull in human subjects by detecting changes in blood hemoglobin concentrations associated with neural activity.

The application in functional mapping of the human cortex is called optical topography (OT), near infrared imaging (NIRI) or functional NIRS (fNIRS). The term optical tomography is used for three-dimensional NIRS. The terms NIRS, NIRI and OT are often used interchangeably, but they have some distinctions. The most important difference between NIRS and OT/NIRI is that OT/NIRI is used mainly to detect changes in optical properties of tissue simultaneously from multiple measurement points and display the results in the form of a map or image over a specific area, whereas NIRS provides quantitative data in absolute terms on up to a few specific points. The latter is also used to investigate other tissues such as, e.g., muscle, breast and tumors.

By employing several wavelengths and time resolved (frequency or time domain) and/or spatially resolved methods blood flow, volume and oxygenation can be quantified. These measurements are a form of oximetry. Applications of oximetry by NIRS methods include the detection of illnesses which affect the blood circulation (e.g., peripheral vascular disease), the detection and assessment of breast tumors, and the optimization of training in sports medicine.

The use of NIRS in conjunction with a bolus injection of indocyanine green (ICG) has been used to measure cerebral blood flow and cerebral metabolic rate of oxygen consumption in neonatal models.

NIRS is starting to be used in pediatric critical care, to help deal with cardiac surgery post-op. Indeed, NIRS is able to measure venous oxygen saturation (SVO2), which is determined by the cardiac output, as well as other parameters (FiO2, hemoglobin, oxygen uptake). Therefore, following the NIRS gives critical care physicians a notion of the cardiac output.

### Positron Emission Tomography (PET)

Positron emission tomography (PET) is a nuclear medicine imaging technique which produces a three-dimensional image or picture of functional processes in the body. The system detects pairs of gamma rays emitted indirectly by a positron-emitting radionuclide (tracer), which is introduced into the body on a biologically active molecule. Images of tracer concentration in 3-dimensional or 4-dimensional space (the 4th dimension being time) within the body are then reconstructed by computer analysis. In modern scanners, this reconstruction is often accomplished with the aid of a CT X-ray scan performed on the patient during the same session, in the same machine.

If the biologically active molecule chosen for PET is fluorodeoxyglucose (FD( ) an analogue of glucose, the concentrations of tracer imaged then give tissue metabolic activity, in terms of regional glucose uptake. Although use of this tracer results in the most common type of PET scan, other tracer molecules are used in PET to image the tissue concentration of many other types of molecules of interest.

To conduct the scan, a short-lived radioactive tracer isotope is injected into the living subject (usually into blood circulation). The tracer is chemically incorporated into a biologically active molecule. There is a waiting period while the active molecule becomes concentrated in tissues of interest; then the research subject or patient is placed in the imaging scanner. The molecule most commonly used for this purpose is FDG, a sugar, for which the waiting period is typically an hour. During the scan a record of tissue concentration is made as the tracer decays.

As the radioisotope undergoes positron emission decay (also known as positive beta decay), it emits a positron, an antiparticle of the electron with opposite charge. The emitted positron travels in tissue fora short distance (typically less than 1 mm, but dependent on the isotope), during which time it loses kinetic energy, until it decelerates to a point where it can interact with an electron. The encounter annihilates both electron and positron, producing a pair of annihilation (gamma) photons moving in approximately opposite directions. These are detected when they reach a scintillator in the scanning device, creating a burst of light which is detected by photomultiplier tubes or silicon avalanche photodiodes (Si APD). The technique depends on simultaneous or coincident detection of the pair of photons moving in approximately opposite direction (it would be exactly opposite in their center of mass frame, but the scanner has no way to know this, and so has a built-in slight direction-error tolerance). Photons that do not arrive in temporal “pairs” (i.e. within a timing-window of a few nanoseconds) are ignored.

### Fluorescence

Methods are provided wherein presence of a composition of the disclosure is detected by an observable change. In one aspect, presence of the composition gives rise to a color change which is observed with a device capable of detecting a specific marker as disclosed herein. For example and without limitation, a fluorescence microscope can detect the presence of a fluorophore that is conjugated to a polynucleotide, which has been functionalized on a nanoparticle.

### Embolic Agents

Administration of an embolic agent in combination with a composition of the disclosure is also contemplated. Embolic agents serve to increase localized drug concentration in target sites through selective occlusion of blood vessels by purposely introducing emboli, while decreasing drug washout by decreasing arterial inflow. Thus, a composition comprising a nanoparticle functionalized with a polynucleotide, wherein the polynucleotide is conjugated to a contrast agent through a conjugation site would remain at a target site for a longer period of time in combination with an embolic agent relative to the period of time the composition would remain at the target site without the embolic agent. Accordingly, in some embodiments, the present disclosure contemplates the use of a composition as described herein in combination with an embolic agent.

In various aspects of the compositions and methods of the disclosure, the embolic agent to be used is selected from the group consisting of a lipid emulsion (for example and without limitation, ethiodized oil or lipiodol), gelatin sponge, tris acetyl gelatin microspheres, embolization coils, ethanol, small molecule drugs, biodegradable microspheres, non-biodegradable microspheres or polymers, and self-assemblying embolic material.

In various embodiments, compositions of the present disclosure are mixed with the embolic agent just prior to administration. The composition/embolic agent mixture may be used alone for nanoembolization, or may be followed by administration of another embolic agent. The term “nanoembolization” as used herein refers to the local delivery of a composition of the disclosure to a target site. Delivery of an embolic agent, in various aspects, can occur before, during, or after, including combinations thereof, the delivery of a composition of the disclosure.

The compositions disclosed herein are administered by any route that permits imaging of the tissue or cell that is desired, and/or treatment of the disease or condition. In one aspect the route of administration is intraarterial administration. Additionally, the composition comprising PN-NP is delivered to a patient using any standard route of administration, including but not limited to orally, parenterally, such as intravenously, intraperitoneally, intrapulmonary, intracardiac, intraosseous infusion (“IO”), subcutaneously or intramuscularly, intrathecally, transdermally, intradermally, rectally, orally, nasally or by inhalation or transmucosal delivery. Direct injection of a composition provided herein is also contemplated and, in some aspects, is delivered via a hypodermic needle. Slow release formulations may also be prepared from the compositions described herein in order to achieve a controlled release of one or more components of a composition as described herein in contact with the body fluids and to provide a substantially constant and effective level of one or more components of a composition in the blood plasma.

It has been shown that intraarterial (IA) delivery alone does now allow for dwell time at a desired target site that is sufficient for efficient uptake of PN-NPs. Thus the addition of an embolic agent allows the blockage of blood flow to a desired site increasing the dwell time of injected PN-NPs which keeps their local concentration high and enhances delivery to tissue. Thus, using IA delivery of NPs combined with an embolic agent greatly increases NP concentration in the vicinity of target cells and limits their distribution throughout the rest of the body, thereby greatly improving NP uptake in targeted cells of interest.

Compositions of the present disclosure comprise ratios of PN-NPs conjugated to a contrast agent and further comprising, in some aspects, an embolic agent. “Ratio,” as used herein, can be a molar ratio, a volume to volume ratio or it can be the number of PN-NPs to the number of embolic agent molecules. One of ordinary skill in the art can determine the ratio to be used in the compositions of the present disclosure.

In some embodiments, the PN-NPs and the embolic agent are present in a ratio of about 1:1 to about 10:1. In further embodiments, the PN-NPs and the embolic agent are present in a ratio of about 2:1 to about 5:1. In one aspect, the PN-NPs and the embolic agent are present in a ratio of about 3:1. The present disclosure contemplates, in various aspects, that compositions of PN-NPs and the embolic agent are present in a ratio of about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 37:1, about 38:1, about 39:1, about 40:1, about 41:1, about 42:1, about 43:1, about 44:1, about 45:1, about 46:1, about 47:1, about 48:1, about 49:1, about 50:1, about 55:1, about 60:1, about 65:1, about 70:1, about 75:1, about 80:1, about 85:1, about 90:1, about 95:1, about 100:1, about 150:1, about 200:1, about 250:1, about 300:1, about 350:1, about 400:1, about 450:1, about 500:1, about 550:1, about 600:1, about 650:1, about 700:1, about 750:1, about 800:1, about 850:1, about 900:1, about 950:1, about 1000:1, about 2000:1, about 5000:1, about 7000:1, about 10000:1 or greater.

In alternative aspects, compositions of PN-NPs and the embolic agent are present in a ratio of about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, about 1:20, about 1:21, about 1:22, about 1:23, about 1:24, about 1:25, about 1:26, about 1:27, about 1:28, about 1:29, about 1:30, about 1:31, about 1:32, about 1:33, about 1:34, about 1:35, about 1:36, about 1:37, about 1:38, about 1:39, about 1:40, about 1:41, about 1:42, about 1:43, about 1:44, about 1:45, about 1:46, about 1:47, about 1:48, about 1:49, about 1:50, about 1:55, about 1:60, about 1:65, about 1:70, about 1:75, about 1:80, about 1:85, about 1:90, about 1:95, about 1:100, about 1:150, about 1:200, about 1:250, about 1:300, about 1:350, about 1:400, about 1:450, about 1:500, about 1:550, about 1:600, about 1:700, about 1:750, about 1:800, about 1:850, about 1:900, about 1:950, about 1:1000, about 1:2000, about 1:5000, about 1:10000 or greater.

In further embodiments, the PN-NPs are approximately lnanomolar (nM) to 10 micromolar (μM), while the embolic agent is in the μM to millimolar (mM) range. Accordingly, in some embodiments, this would yield PN-NP:embolic agent ratios of about 1:1, about 1:10, about 1:100, about 1:1000, about 1:10,000 or higher.

### Target Site Identification and Composition Delivery

Provided herein are methods of delivering a contrast agent to a cell comprising contacting the cell with a composition of the disclosure under conditions sufficient to deliver the contrast agent to the cell. Following delivery of the composition, in some aspects the method further comprises the step of detecting the contrast agent. Detecting the contrast agent is performed by any of the methods known in the art, including those described herein.

In a specific embodiment, the contrast agent is detected using an imaging procedure, and in various aspects, the imaging procedure is selected from the group consisting of MRI, CT, and fluorescence.

In some embodiments, the methods further comprise a detectable marker attached to a polynucleotide that is functionalized to a nanoparticle. A further aspect of the method, then, is detecting the detectable marker that is attached to the polynucleotide. These aspects are discussed further below.

Methods provided also include those wherein a composition of the disclosure is locally delivered to a target site. Once the target site has been identified, a composition of the disclosure is delivered, in one aspect, intraarterially. In another aspect, a composition of the disclosure is delivered intravenously. Target cells for delivery of a composition of the disclosure are, in various aspects, selected from the group consisting of a cancer cell, a stem cell, a T-cell, and a β-islet cell.

Target site identification is performed, in some aspects, by interventional radiology. For example and without limitation, an IR procedure is performed in which a catheter is advanced into the artery directly supplying a tumor to be treated under image guidance. Perfusion of the tumor is confirmed, then the PN-NP/embolic agent composition is injected, with or without injection of an additional embolic agent. In aspects where an additional embolic agent is administered, the additional embolic agent can be part of the composition or, in some aspects, can be administered separately from the composition. In aspects where the additional embolic agent is administered separately from the composition, it is contemplated that the additional embolic agent can be administered before or after the composition.

Intraarterial drug delivery, pioneered by the field of interventional radiology (IR), has been used extensively in the minimally invasive treatment of a wide variety of diseases including solid tumors. IR physicians are able to catheterize the blood supply directly feeding a solid tumor and deliver relatively high doses of chemotherapeutics while limiting the systemic side effects of such drugs. This process is followed by the administration of an embolic agent to block blood flow to the tumor starving it of nutrients and increasing the dwell time of injected therapeutics, keeping the local concentration of chemotherapeutic high. Using IA delivery of nanoparticles, either in conjunction with an embolic agent or followed by injection of an embolic agent, greatly increases NP concentration in tumor cells and limits their distribution throughout the rest of the body, thus greatly improving their uptake in cancer cells.

For nanoembolization, a vascular catheter is advanced superselectively under fluoroscopic guidance into a tumor's feeding artery. Therapeutic nanoparticles are then infused through the catheter, along with embolic agents, with the goal of maximizing intratumoral drug concentration. This material is used, for example and without limitation, for the treatment of cancer as described above, the delivery of therapeutic agents for tissue regeneration or growth of tissue, or for the delivery of molecularly targeted contrast agents.

Image-guided nanoembolization takes advantage of a number of imaging modalities including MRI, CT, X-Ray DSA, X-ray attenuation or ultrasound to guide catheter placement, confirm target cell perfusion, and deliver NPs locally.

In various aspects, the target site is a site of pathogenesis.

In some aspects, the site of pathogenesis is cancer. In various aspects, the cancer is selected from the group consisting of liver, pancreatic, stomach, colorectal, prostate, testicular, renal cell, breast, bladder, ureteral, brain, lung, connective tissue, hematological, cardiovascular, lymphatic, skin, bone, eye, nasopharyngeal, laryngeal, esophagus, oral membrane, tongue, thyroid, parotid, mediastinum, ovary, uterus, adnexal, small bowel, appendix, carcinoid, gall bladder, pituitary, cancer arising from metastatic spread, and cancer arising from endodermal, mesodermal or ectodermally-derived tissues.

In some embodiments, the site of pathogenesis is a solid organ disease. In various aspects, the solid organ is selected from the group consisting of heart, liver, pancreas, prostate, brain, eye, thyroid, pituitary, parotid, skin, spleen, stomach, esophagus, gall bladder, small bowel, bile duct, appendix, colon, rectum, breast, bladder, kidney, ureter, lung, and a endodermally-, ectodermally- or mesodermally-derived tissues.

Methods provided further contemplate a second delivery of a composition as described herein is performed. In various aspects, the second delivery of the composition is administered after 24 hours. Methods including one or more subsequent administrations include those wherein the composition is administered for again about daily, about weekly, about every other week, about monthly, about every 6 weeks, or about every other month. Shorter time frames are also contemplated, wherein a subsequent delivery of the composition occurs within about a minute, about an hour, more than one day, about a week, or about a month following an initial administration of the composition.

In some embodiments, the second delivery of the composition occurs within about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 30 minutes, about 40 minutes, about 50 minutes, about 60 minutes, about 8 hours, about 2 days, about 3 days, about 4 days, about 5 days, about 6 days, about 10 days, about 15 days, about 20 days, about 25 days or more following an initial administration of the composition.

These schedules, in various aspects, would follow the chemotherapy paradigm of treating patients with a series of doses, separated in time to optimize therapeutic benefit, while minimizing toxicity. Each single dosing would, in various aspects, take minutes to hours to deliver. In some aspects, an administration schedule comprises continuous intraarterial administration using an implantable catheter that occurs, in various aspects, over a time course of days to weeks.

### Detectable Marker

Methods are provided wherein a polynucleotide as described herein is detected by a detectable marker. In one aspect, presence of the polynucleotide gives rise to a color change which is observed with a device capable of detecting a specific marker as disclosed herein. For example and without limitation, a fluorescence microscope can detect the presence of a fluorophore that is conjugated to a polynucleotide, which has been functionalized on a nanoparticle. In various aspects and as described above, when modified with a fluorophore, the PN-NPs as described herein can be used as multimodal contrast agents where fluorescence microscopy indicates that the particles localize in the perinuclear region inside cells. In further aspects, surface-enhanced Raman scattering (SERS) can be used to detect the presence of the nanoparticle in a composition as described herein. In still further aspects, electron microscopy is used to detect the presence of the nanoparticle in a composition as described herein.

It will be understood that a marker contemplated will include any of the fluorophores described herein as well as other detectable markers known in the art. For example, markers also include, but are not limited to, redox active probes, other nanoparticles, and quantum dots, as well as any marker which can be detected using spectroscopic means, i.e., those markers detectable using microscopy and cytometry. In various aspects, isotopes are contemplated as a general method of identifying the location of embolized material. A luminophore can also be used in a general method of identifying the location of embolized material.

Suitable fluorescent molecules are also well known in the art and include without limitation 1,8-ANS (1-Anilinonaphthalene-8-sulfonic acid), 1-Anilinonaphthalene-8-sulfonic acid (1,8-ANS), 5-(and-6)-Carboxy-2′,7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin) Atto 647, BCECF pH 5.5, BCECF pH 9.0, BFP (Blue Fluorescent Protein), BO-PRO-1-DNA, BO-PRO-3-DNA, BOBO-1-DNA, BOBO-3-DNA, BODIPY 650/665-X, MeOH, BODIPY FL conjugate, BODIPY FL, MeOH, Bodipy R6G SE, BODIPY R6G, MeOH, BODIPY TMR-X antibody conjugate pH 7.2, Bodipy TMR-X conjugate, BODIPY TMR-X, MeOH, BODIPY TMR-X, SE, BODIPY TR-X phallacidin pH 7.0, BODIPY TR-X, MeOH, BODIPY TR-X, SE, BOPRO-1, BOPRO-3, Calcein, Calcein pH 9.0, Calcium Crimson, Calcium Crimson Ca2+, Calcium Green, Calcium Green-1 Ca2+, Calcium Orange, Calcium Orange Calif.2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), C₁-NERF pH 2.5, CI-NERF pH 6.0, Citrine, Coumarin, Cy 2, Cy 3, Cy 3.5, Cy 5, Cy 5.5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, DiI, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, Ethidium Bromide, Ethidium homodimer, Ethidium homodimer-1-DNA, eYFP (Enhanced Yellow Fluorescent Protein), FDA, FITC, FITC antibody conjugate pH 8.0, FlAsH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Ca, Fura-2 Ca2+, Fura-2, high Ca, Fura-2, no Co, GFP(S65T), HcRed, Hoechst 33258, Hoechst 33258-DNA, Hoechst 33342, Indo-1 Ca2+, Indo-1, Ca free, Indo-1, Ca saturated, JC-1, JC-1 pH 8.2, Lissamine rhodamine, LOLO-1-DNA, Lucifer Yellow, CH, LysoSensor Blue, LysoSensor Blue pH 5.0, LysoSensor Green, LysoSensor Green pH 5.0, LysoSensor Yellow pH 3.0, LysoSensor Yellow pH 9.0, LysoTracker Blue, LysoTracker Green, LysoTracker Red, Magnesium Green, Magnesium Green Mg2+, Magnesium Orange, Marina Blue, mBanana, mCherry, mHoneydew, MitoTracker Green, MitoTracker Green FM, MeOH, MitoTracker Orange, MitoTracker Orange, MeOH, MitoTracker Red, MitoTracker Red, MeOH, mOrange, mPlum, mRFP, mStrawberry, mTangerine, NBD-X, NBD-X, MeOH, NeuroTrace 500/525, green fluorescent Nissl stain-RNA, Nile Blue, EtOH, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, PicoGreen dsDNA quantitation reagent, PO-PRO-1, PO-PRO-1-DNA, PO-PRO-3, PO-PRO-3-DNA, POPO-1, POPO-1-DNA, POPO-3, Propidium Iodide, Propidium Iodide-DNA, R-Phycoerythrin pH 7.5, ReAsH, Resorufin, Resorufin pH 9.0, Rhod-2, Rhod-2 Ca2+, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodaminen Green pH 7.0, Rhodol Green antibody conjugate pH 8.0, Sapphire, SBFI-Na+, Sodium Green Na+, Sulforhodamine 101, EtOH, SYBR Green I, SYPRO Ruby, SYTO 13-DNA, SYTO 45-DNA, SYTOX Blue-DNA, Tetramethylrhodamine antibody conjugate pH 8.0, Tetramethylrhodamine dextran pH 7.0, Texas Red-X antibody conjugate pH 7.2, TO-PRO-1-DNA, TO-PRO-3-DNA, TOTO-1-DNA, TOTO-3-DNA, TRITC, X-Rhod-1 Ca2+, YO-PRO-1-DNA, YO-PRO-3-DNA, YOYO-1-DNA, and YOYO-3-DNA.

In yet another embodiment, two types of fluorescent-labeled polynucleotides attached to two different nanoparticles can be used. This may be useful, for example and without limitation, to track two different cell populations.

Methods of labeling polynucleotides with fluorescent molecules and measuring fluorescence are well known in the art.

### Therapeutic Agents

Therapeutic agents as disclosed herein below are contemplated for use in conjunction with a composition of the disclosure. In some aspects, the therapeutic agent is administered in combination with a composition of the disclosure that has both imaging as well as gene regulatory capabilities. In some of these aspects, a polynucleotide functionalized on the nanoparticle of the composition further comprises a domain that affects the uptake efficiency of the functionalized nanoparticle. In further embodiments, the composition and the therapeutic agent are delivered with an embolic agent as described herein.

Compositions of the disclosure are contemplated for use in delivery to a cell. In various aspects, the cell is a cancer cell or a stem cell. It is therefore contemplated that a therapeutic agent is likewise administered in conjunction with the composition. For example and without limitation, in certain instances it is advantageous to administer a chemotherapeutic agent in conjunction with a composition that is, in some aspects, targeting a cancer cell.

Likewise, one of skill in the art would also understand the benefit of administering a growth factor in conjunction with a composition that, in other aspects, targets a stem cell. In these aspects, it is contemplated that a composition of the disclosure is administered to a cell which is then delivered to a site in the recipient. In other aspects, the composition comprises a targeting moiety that directs the composition to a specific cell, tissue, organ or other desired site. In some of these aspects the polynucleotide that is functionalized on the nanoparticle in the composition further comprises a detectable marker as described herein.

A therapeutic agent, in some embodiments, is co-administered with a composition of the disclosure. Alternatively, a therapeutic agent may be delivered before or after the administration of a composition of the disclosure. In various aspects, the therapeutic agent is delivered minutes, hours or days either before or after the administration of a composition of the disclosure. It is also contemplated that, in various aspects, more than one therapeutic agent is administered. In these aspects, the more than one therapeutic agents are administered at the same time. In further aspects, the more than one therapeutic agents are administered sequentially. The clinician of ordinary skill in the art can determine the administration schedule of a given therapeutic agent or combination of therapeutic agents.

Accordingly, in some embodiments, a composition of the present disclosure further comprises a therapeutic agent. In some aspects, the therapeutic agent is associated with the nanoparticle. In other aspects, the therapeutic agent is co-administered with the PN-NP, but is separate from the PN-NP composition. In further aspects, the therapeutic agent is administered before the administration of the PN-NP composition, and in still further aspects, the therapeutic agent is administered after the administration of the PN-NP composition. One of ordinary skill in the art will understand that multiple therapeutic agents in multiple combinations can be administered at any time before, during or after administration of the PN-NP composition. In addition, repeated administration of a therapeutic agent is also contemplated.

In an embodiment of the invention, the therapeutic agent is selected from the group consisting of a protein, peptide, a chemotherapeutic agent, a small molecule, a radioactive material, and a polynucleotide.

Protein therapeutic agents include, without limitation peptides, enzymes, structural proteins, receptors and other cellular or circulating proteins as well as fragments and derivatives thereof, the aberrant expression of which gives rise to one or more disorders. Therapeutic agents also include, as one specific embodiment, chemotherapeutic agents. Still other therapeutic agents include polynucleotides, including without limitation, protein coding polynucleotides, polynucleotides encoding regulatory polynucleotides, and/or polynucleotides which are regulatory in themselves. Therapeutic agents also include, in various embodiments, a radioactive material.

In various aspects, protein therapeutic agents include cytokines or hematopoietic factors including without limitation pleiotrophin, IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2a, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor 13 binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

In other aspects, chemotherapeutic agent include, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinium coordination complexes such as cisplatin and carboplatin, anthracenediones such as mitoxantrone, substituted urea such as hydroxyurea, methylhydrazine derivatives including N-methylhydrazine (M1H) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

The term “small molecule,” as used herein, refers to a chemical compound, for instance a peptidometic or polynucleotide that may optionally be derivatized, or any other low molecular weight organic compound, either natural or synthetic. Such small molecules may be a therapeutically deliverable substance or may be further derivatized to facilitate delivery.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

Polynucleotide therapeutic agents include, in one aspect and without limitation, those which encode therapeutic proteins described herein and otherwise known in the art, as well as polynucleotides which have intrinsic regulatory functions. Polynucleotides that have regulatory functions have been described herein above and include without limitation RNAi, antisense, ribozymes, and triplex-forming polynucleotides, each of which have the ability to regulate gene expression. Methods for carrying out these regulatory functions have previously been described in the art (Dykxhoom D M, Novina C D and Sharp P A, Nature Review, 4: 457-467, 2003; Mittal V, Nature Reviews, 5: 355-365, 2004).

It will be appreciated that, in various aspects, a therapeutic agent as described herein is attached to the nanoparticle.

### EXAMPLES

### Example 1

### Preparation of the Nanoconjugate

Nanoparticles. Citrate-stabilized AuNPs (13±1.0 nm diameter) were prepared as described previously. AuNPs of 30 nm in diameter were purchased from Ted Pella Inc (USA). Polynucleotides were synthesized on an Expedite 8909 Nucleotide Synthesis System (ABI) by standard solid-phase phosphoramidite synthesis techniques. All bases and reagents were purchased from Glen Research. The polynucleotides were purified using reverse-phase high-performance liquid chromatography (RP-HPLC) using a Varian Microsorb C18 column (10 mm, 300 mm) with 0.03 M triethylammonium acetate (TEAA), at pH 7.0, and a 1.0% per min gradient of 95% CH₃CN/5% 0.03 M TEAA at a flow rate of 3 ml/min while monitoring the UV signal of DNA at 254 nm. After purification, the polynucleotides were lyophilized and stored at −78° C. until use. Before nanoparticle conjugation, the 3-disulfide functionality was reduced with Dithiothreitol (DTT) following published procedures.

Synthesis of amine-modified polynucleotides. Polynucleotides (3′ SH-T9TTTNH₂ TTT NH₂TTT NH₂TTTNH₂TTTNH₂ 5′: SEQ ID NO: 1) were prepared by the conventional phosphoamidite method on 3′-thiol modifier C6 controlled pore glass supports (1.0 μmol) using an Expedite 8909 Nucleotide Synthesis System (ABI). To incorporate the amino group into the polynucleotides, amino-modifier C6 dT phosphoamidite (TNH₂) (Glen research, USA) were used during the DNA synthesis. After automated synthesis, the glass supports were treated with a mixture of saturated 30% ammonia (aq.) at 55° C. for 16 hours. Detached and deprotected polynucleotides were evaporated to dryness, dissolved in water, and purified by RP-HPLC. The polynucleotides were characterized by MALDI-MS. The concentrations of polynucleotides were determined by monitoring the absorbance at 260 nm UV-Cary 5000 spectrophotometer.

Synthesis of Azido-modified Polynucleotides.

Azido-modified polynucleotide can be obtained by conjugating post-synthesis of an amino-modified polynucleotide with an azide N-hydroxysuccinimide (NHS) ester, azidobutyrate NHS Ester (Glen Research, USA). Lyophilized amino-modified polynucleotide (1 μmol) was dissolved in 0.5 mL of 0.1M Na₂CO₃/NaHCO₃ buffer (pH 8.5). To this solution, excess of azide N-hydroxysuccinimide (NHS) ester (5 mg) in 100 μl of DMSO was added. The resulting mixture was incubated overnight at room temperature, purified by RP-HPLC and characterized by MALDI-TOF MS.

Synthesis of DNA-Gd(III) Conjugates

The Gd(III)-modified polynucleotides was synthesized by coupling an azido-modified polynucleotide and hexynyl-modified Gd(III)chelate MRI contrast agent through a click chemistry approach. To 950 μL Of 0.20 M aqueous NaCl Tris-hydroxypropyl triazolyl ligand (2.0 μmmol), sodium ascorbic acid (2.0 μmol) and copper (II) sulphate pentahydrate (0.40 vitriol), Gd(III)-chelate (10 mg) were added sequentially. The above solution was added to lyophilized azido-modified polynucleotide (1.0 μmol) and incubated for 2.0 hours to allow for the click-chemistry ligation to occur.

Preparation of DNA-Gd(III)-AuNP conjugates (Scheme 1).

The 13 nm AuNPs were synthesized and functionalized with polynucleotides according to previously reported methods. 30 nm AuNPs were purchased from Ted Pella (Redding, Calif.). AuNPs were functionalized with alkanethiol-modified polynucleotides. Prior to use, the disulfide functionality on the polynucleotides was cleaved by addition of DTT to lyophilized DNA and the resultant mixture incubated at room temperature for 2.0 hours (0.1 M DTT, 0.18 M phosphate buffer (PB), pH 8.0). The cleaved polynucleotides were purified using a NAP-5 column. Freshly cleaved polynucleotides were added to AuNPs (10D/1.0 mL), and the concentrations of PB and sodium dodecyl sulfate (SDS) were brought to 0.01 M and 0.01%, respectively. The polynucleotide/AuNPs solution was allowed to incubate at room temperature for 20 min. The concentration of NaCl was increased to 0.10 M using 2.0 M NaC1, 0.01 M PBS while maintaining an SDS concentration of 0.01%. The final mixture was brought to 0.10 M NaCl over 24 hours and shaken for an additional 24 hours to complete the process.

Accordingly, NP conjugates were prepared by reacting citrate stabilized gold nanoparticles with thiol-labeled 24-mer poly dT polynucleotides (polydT) DNA polynucleotides were synthesized on a solid support with post-modification carried out in solution. The poly dT contained five conjugation sites (hexylamino labeled dT groups conjugated with a cross linker, azidobutyrate N-hydroxysuccinimideester) for covalently attaching Gd(III) complexes through click chemistry. Click chemistry has proven to be an efficient method for preparing Gd(III)-based MR contrast agents with high synthetic yields and increased relaxivity [Song et al., J. Am. Chem. Soc. 130: 6662 (2008)].

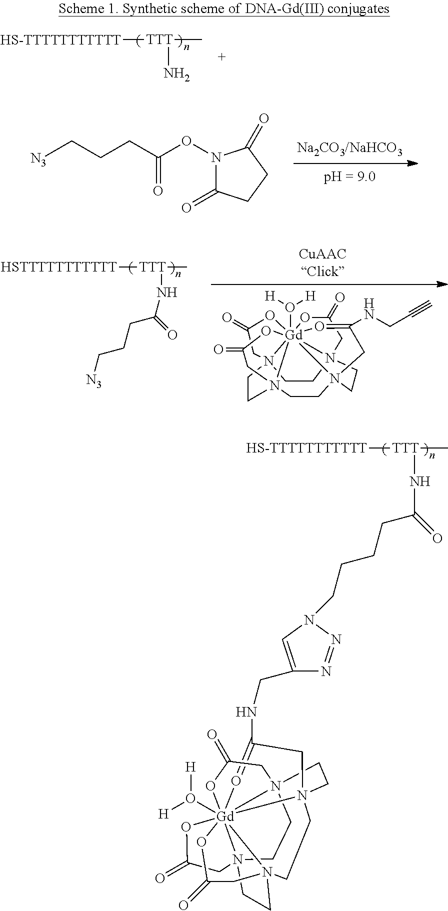

### Example 2

After purification by RP-HPLC, the DNA-Gd(III) conjugates were characterized by MALDI-MS, which confirmed formation of the conjugates. The DNA-Gd(III) conjugates were then immobilized on citrate stabilized gold nanoparticles (AuNPs) following literature procedures used to make the analogous Gd(III)-free NPs to yield DNA-Gd(III)-AuNPs (Scheme 2, below) [Storhoff et al., J. Am. Chem. Soc. 120:1959 (1998)]. Excess DNA-Gd(III) was removed by repeated centrifugation and resuspension of the NPs until the supernatant was free of Gd(III). When suspended in aqueous solution, the NP conjugates appear deep red in color due to the plasmon resonance of the Au at 520 nm, and they are stable for months at room temperature. Cy3-labelled DNA polynucleotides (5′-Cy3-TTTTTTTTTTTTTTTTTTTTTTTT-5H-3′: SEQ ID NO: 2, shown in Scheme 2) were synthesized for fluorescence microscopy and flow cytometry to confirm cell uptake and labeling efficiency, respectively.

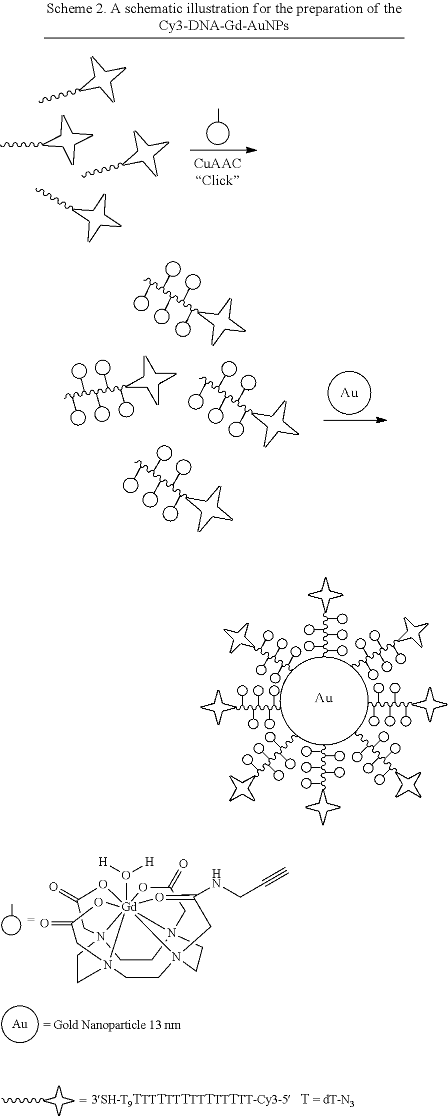

Relaxivity (r₁). To determine relaxivity, a stock solution of DNA-Gd(III)-AuNPs was prepared in 200 μL of water, and diluted with 20 uL of water after each T₁ acquisition. Ts were determined at 60 MHz (1.41T) and 37° C. using an inversion recovery pulse sequence on a Bruker mq60 minispec using 4 averages, 15 second repetition time, and 10 data points (Bruker Canada; Milton, Ontario, Canada). The starting and final Gd(III) concentrations of the solutions were determined using ICP-MS. The inverse of the longitudinal relaxation time (1/T₁, s⁻¹) was plotted against Gd(III) concentration (mM) and fitted to a straight line. Lines were fit with R²>0.99.

The relaxation efficiency of these newly synthesized MR contrast agent conjugates was determined by taking the slope of a plot of the measured 1/T₁ as a function of Gd(III) concentration. The resultant relaxivity, r₁, of the Gd(III) complex after conjugation to DNA was determined to be 8.7 mM⁻¹ s⁻¹ at 37° C. in water at 60 MHz (1.41T). This represents a two-fold increase over the unconjugated Gd(III) complex (3.2 mM⁻¹s⁻¹, Table 1). This doubling in relaxivity is consistent with Soloman-Bloomberg-Morgan theory where decreases in rotational correlation time, τᵣ, result in increases in r₁ [Merbach et al., Editors, The Chemistry of Contrast Agents in Medical Magnetic Resonance Imaging, 1st ed., Wiley, New York, 2001; Giljohann et al., Nano Lett. 7: 3818 (2007)].

TABLE 1
Relaxivities (r₁s) of Gd(III) complexes and conjugates
at 60 MHz and 600 MHz.

r₁(mM⁻¹s⁻¹)

| | 60 MHz | 600 MHz |
|---|---|---|
| | (1.41T)^{a} | (14.1T)^{b} |
| DOTA-Gd(III) | 3.2^{c} | 2.2 |
| DNA-Gd(III) | 8.7 | — |
| 13 nm DNA-Gd(III)-AuNP/ionic | 16.9 | 5.1 |
| 13 nm DNA-Gd(III)-AuNP/particle | 4225 | 1275 |

^{a}Measured in pure water at 37° C.
^{b}Measured in cell media at 25° C.
^{c}Data taken from [Merbach et al., Editors, The Chemistry of Contrast Agents in Medical Magnetic Resonance Imaging, 1st ed., Wiley, New York, 2001.]

It is important to note that the relaxivity of Gd(III) increases further when DNA-Gd(III) is immobilized on the surface of AuNPs through gold thiol linkages. Two different sizes of AuNPs (13 and 30 nm) have been examined and it was found that the ionic relaxivity [per Gd(III)] was 16.9 mM⁻¹ s⁻¹ for 13 nm DNA-Gd(III)-AuNPs and 20.0 mM⁻¹ s⁻¹ for 30 nm DNA-Gd(III)-AuNPs.

Inductively Coupled Plasma-Mass Spectrometry (ICP-MS).

Quantitation of Au and Gd was accomplished using ICP-MS of acid digested samples. Specifically, 50 μL of TraceSelect nitric acid (>69%, Sigma, St. Louis, Mo.) was added to cell suspensions or media and placed at 65° C. for at least 4 hours to allow for complete sample digestion. 50 μL of TraceSelect HCl (fuming 37%, Sigma, St. Louis, Mo.) was then added to each sample for long term sample stability and elimination of matrix effects. Nanopure H₂O and multi-element internal standard were added to produce a solution of 1.5% nitric acid (v/v), 1.5% HCl (v/v) and 5.0 ng/mL internal standard up to a total sample volume of 3 mL. Individual Au and Gd(III) elemental standards were prepared at 0.500, 1.00, 5.00, 10.0, 25.0, 50.0, 100, and 250 ng/mL concentrations with 1.5% nitric acid (v/v), 1.5% HCl (v/v) and 5.0 ng/mL internal standards up to a total sample volume of 10 mL.

ICP-MS was performed on either a computer-controlled (Plasmalab software) Thermo (Thermo Fisher Scientific, Waltham, Mass.) PQ ExCell ICP-MS equipped with a CETAC 500 autosampler or a computer-controlled (Plasmalab software) Thermo X series II ICP-MS equipped with an ESI (Omaha, Nebr., USA) SC-2 autosampler. Each sample was acquired using 1 survey run (10 sweeps) and 3 main (peak jumping) runs (100 sweeps). The isotopes selected were ¹⁹⁷Au, ^{156,157}Gd and ¹¹⁵In, ¹⁶⁵Ho, and ²⁰⁹Bi (as internal standards for data interpolation and machine stability).

The degree of conjugation of the chelates to the AuNP surface, the Gd(III) to Au ratio was determined via ICP-MS where the 13 nm AuNPs have 50±5 strands of DNA-Gd(III) per NP [400±25 Gd(III) per NP] and the 30 nm AuNPs have 100±10 strands per NP [500±50 Gd(III) per NP]. These calculations were based on the assumption that there are 65,800 Au atoms per 13 nm AuNP, and 800,650 Au atoms per 30 nm AuNP (numbers were determined by geometric arguments and the crystal structure of bulk gold). Taking into account the loading of Gd(III) per particle, the 13 nm DNA-Gd(III)-AuNPs exhibited a relaxivity of approximately 4225 mM⁻¹ s⁻¹ per particle (Table 1).

MR imaging and T₁ Analysis.

14.1T MR imaging and T₁ measurements were performed on a General Electric/Bruker Omega 600WB 14.1T imaging spectrometer fitted with accustar shielded gradient coils at 25° C. For solution phantoms, 50 u1_, of 60, 40 and 20 μM Gd(III) (DOTA-Gd(III) and Gd(III)-AuNP) in complete cell media were added to flame-sealed 5¾″ Pasteur pipettes and centrifuged at 4.0° C. and 100×g for 5.0 minutes. Capillaries were then placed in a custom-made glass capillary holder and imaged in a 20 mm birdcage coil. For cell phantoms, approximately 1.5×10⁶ NIH/3T3 cells were incubated with 20 or 5.0 μM ([Gd(III)]) Gd(III)-AuNP or DOTA-Gd(III) for 24 hours, rinsed two times with DPBS, and harvested with trypsin. After addition of complete media (1.0 mL total volume) cells were added to flame-sealed 5¾″ Pasteur pipettes and centrifuged at 4.0° C. and 100×g for 5.0 minutes. Capillaries were then placed in a custom-made glass capillary holder and imaged in a 10 mm birdcage coil. Spin lattice relaxation times (T₁) were measured using a saturation recovery pulse sequence with static TE (10.18 ms) and variable TR (350, 500, 750, 1000, 1500, 2500, 4000, 7500, 15000 ms) values. Imaging parameters were as follows: field of view (FOV)=10×10 mm² (20×20 mm² for solution phantoms), matrix size (MTX)=256×256, number of axial slices=4 (3 for solution phantoms), slice thickness (SI)=1.0 mm, and averages (NEX)=6 (2 for solution phantoms). T₁ analysis was carried out using the image sequence analysis tool in Paravision 3.0.2 software (Bruker BioSpin, Billerica, Mass., USA) with monoexponential curve-fitting of image intensities of selected regions of interest (ROIs) for each axial slice.

3T MR images were acquired on a Siemens 3T TIM Trio imaging system using a 35 mm diameter mouse body coil. 200 uL samples of 60, 40 and 20 μM Gd(III) (DOTA-Gd(III) and Gd(III)-AuNP) solutions were placed in wells of a 96-well plate alongside 200 uL samples of unlabeled AuNP and water. Samples were imaged at ambient temperature (approximately 25° C.) using a T₁-weighted spin echo sequence with TR=500 ms, TE=11 ms, FOV=27×100 mm², imaging matrix size=192×259, slice thickness=2 mm, and 4 signal averages.

T₁-weighted MR images of the DNA-Gd(III)-AuNPs in solution phantoms were acquired at 3T and 14.1T at 25° C. The images show that at each concentration [60 μM, 40 20 μM Gd(III)], DNA-Gd(III)-AuNPs appear significantly brighter than DOTA-Gd(III) samples at the same concentration at both field strengths. T₁ analysis at 14.1T reveals a 52% reduction in T₁ for DNA-Gd(III)-AuNPs [60 μM Gd(III)] versus a 31% reduction for DOTA-Gd(III). The image-based r₁ (at 14.1T) of DNA-Gd(III)-AuNP is 5.1 mM⁻¹ s⁻¹ whereas the r₁ of DOTA-Gd(III) is 2.1 mM⁻¹ s⁻¹ (Table 1).

General Cell Culture.

NIH/3T3 and HeLa cells were purchased from American Type Culture Collection (ATCC, Manassas, Va., USA). Media, Dulbecco's phosphate buffered saline (DPBS), and 0.25% trypsin/EDTA solutions were purchased from Invitrogen (Carlsbad, Calif., USA). All corning brand cell culture consumables (flasks, plates, and serological pipettes) were purchased from Fisher Scientific (Pittsburgh, Pa.). NIH/3T3 cells were cultured using DMEM (with 4 mM L-glutamine modified to contain 4.5 g/L glucose and 1.5 g/L sodium carbonate) supplemented with 10% CBS (ATCC). HeLa cells were cultured using EMEM (with Earle's balanced salt solution and 2.0 mM L-glutamine modified to contain 1.0 mM sodium pyruvate, 0.1 mM nonessential amino acids, and 1.5 g/L sodium bicarbonate) supplemented with 10% FBS (Mediatech, Manassas, Va., USA). All experiments were done in the aforementioned cell-specific media in a 5.0% CO₂ incubator operating at 37° C. NIH/3T3 and HeLa cells were harvested using a 0.25% trypsin/EDTA solution. All compounds/nanoparticles incubation, leaching, and harvesting were carried out at 37° C. in a 5.0% CO₂ incubator unless otherwise specified.

### Flow Cytometry

Cell Counting and Percent Cell Viability Determination Using a Guava EasyCyte Mini Personal Cell Analyzer (PCA) Flow Cytometry System. Cells were counted and percent cell viability determined via a Guava EasyCyte mini personal cell analyzer (Guava Technologies, Hayward, Calif., USA). Specifically, after cell harvesting an aliquot (10 or 20 μL) of the cell suspensions were mixed with Guava ViaCount reagent (final sample volume of 200 μL) and allowed to stain at room temperature for at least 5 minutes (no longer than 20 minutes). Stained samples were then vortexed for 5 seconds, after which cells were counted and percent cell viability determined via manual analysis using the ViaCount software module. For each sample, 1000 events were acquired with dilution factors that were determined based upon optimum machine performance (approximately 50-200 cells/μL). Instrument reproducibility was assessed daily using GuavaCheck Beads and following the manufacturer's suggested protocol using the Daily Check software module.

Assess Percentage of Cell Labeling with Cy₃ Labeled Gd-AuNPs by Flow Cytometry.

The uptake Cy3-DNA-Gd(III)-AuNPs was assessed using flow cytometry (BD LSR, BD Biosciences, San Jose, Calif.). NIH/3T3 cells were incubated with 0.15 nM Cy3-DNA-Gd(III)-AuNPs for 4.0 hours. Cells were then washed with PBS three times, followed by incubation with 2.5 μg/ml of Hoechst 33342 (nuclear counterstain) for 20 min at room temperature in dark. Following another PBS wash to remove excess Hoescht, cells were trypsinized and centrifuged at 200×g and 25° C. to remove excess trypsin/EDTA. Cells were then resuspended in 0.5 mL of PBS and assessed using flow cytometry. Dot plots were gated on FSC/SSC properties of NIH/3T3 cells to exclude free fluorescent labeled nanoparticles. Data were analyzed using BD FACSDiVa™ based software. Quadrant markers were set accordingly with controls.

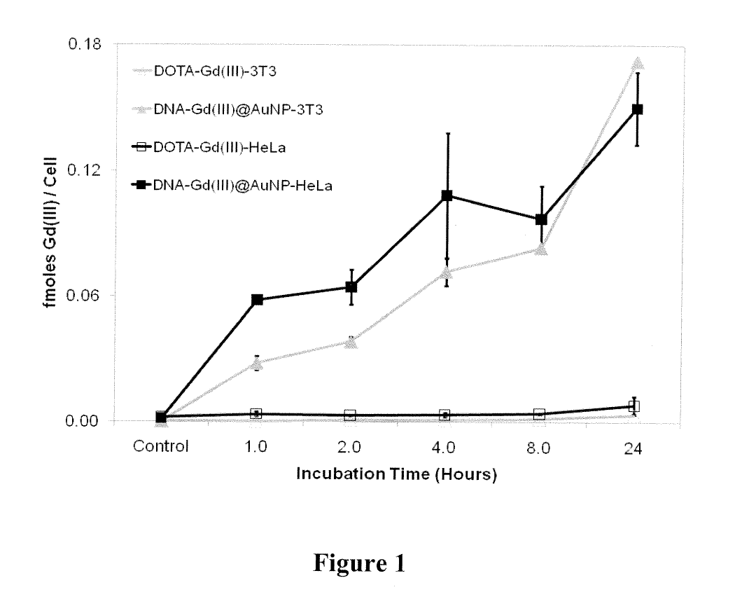

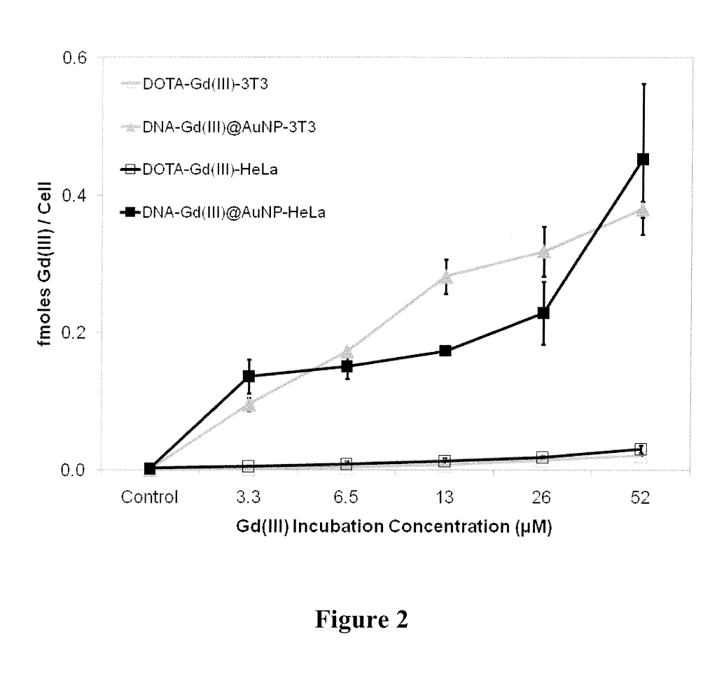

To determine the efficacy of cellular uptake, NIH/3T3 and HeLa cells were labeled with increasing concentrations of DNA-Gd(III)-AuNPs or DOTA-Gd(III) for different amounts of time. Following agent incubation, cells were rinsed with DPBS, counted and then percent viability was assessed via flow cytometry. Gd(III) and Au content were determined via ICP-MS of acid digested samples. The cellular uptake of DNA-Gd(III)-AuNPs was both time- and concentration-dependent (FIGS. 1 and 2). At all concentrations the Gd(III) uptake was >50-fold higher for DNA-Gd(III)-AuNPs than DOTA-Gd(III). On average, cells take up 10⁶-10⁷ Gd(111) atoms per cell using uM Gd(I11) incubation concentrations. Previously, reports have suggested that at least 10⁷-10⁹ Gd(III) atoms per cell are necessary to produce detectable contrast enhancement. These reports, however, use mM incubation concentrations of Gd(III). The nanoparticle concentration is over two orders of magnitude lower since each particle contains approximately 50 strands of DNA-Gd conjugates.

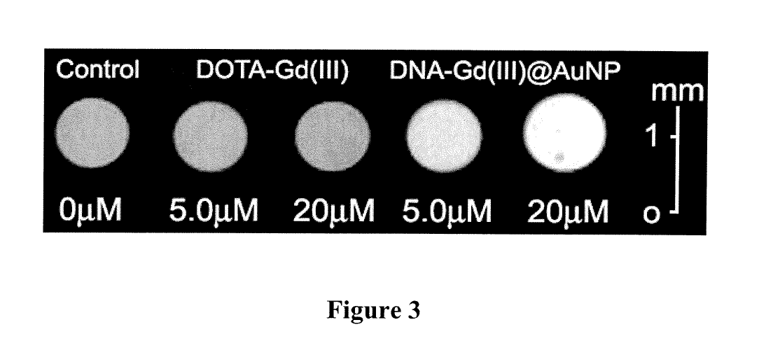

To demonstrate that uM Gd(III) incubation concentrations of DNA-Gd(III)-AuNP conjugates were sufficient to produce significant T₁-weighted contrast enhancement of small cell populations, cells were labeled and imaged at 14.1 T. Specifically, NIH/3T3 cells were incubated with 5.0 μM or 20 μM [Gd(III) concentration] of DOTA-Gd(III) or DNA-Gd(III)-AuNP for 24 hours. T₁ weighted MR images of cell pellets were acquired in 1.0 mm diameter glass capillaries, each containing approximately 10⁶ cells (FIG. 3). T₁ analysis revealed a 43% and 29% T₁ reduction with 20 and 5.0 μM DNA-Gd(III)-AuNP labeled cell pellets, respectively. Cell pellets incubated with DOTA-Gd(III) at either concentration showed no significant difference from control cell pellets. It is believed that these results represent the lowest reported incubation concentration of a Gd(III) complex or conjugate to produce greater than 40% reduction of T₁ in cell pellets [Biancone et al., NMR in biomedicine 20: 40 (2007)].

For comparison, MRI has been applied to tracking Gd(III) labeled β-islets for transplantation and stem cell migration with DOTA-Gd(III) with incubation concentrations ranging from 20-50 mM [Crich et al., Mag. Reson. Med. 51: 938 (2004)]. It is noted that on average the cells internalize approximately 10⁵ Gd(III)— conjugates/cell, which is 2 orders of magnitude higher than citrate-stabilized AuNPs of the same size. A 1000-fold decrease in Gd(III) incubation concentration to obtain essentially the same contrast enhancement is reported herein. It was found that efficient delivery and accumulation of Gd(III) complexes is critical for improving the detection limit for high resolution (concurrently high magnetic field) cellular imaging.

The Gd(III)-DNA-AuNP conjugates are resistant to nuclease degradation which is important for long term cell tracking [Modo et al., Editors, Molecular and Cellular MR Imaging, CRC Press, FL, 2007]. It was determined (via ICP-MS) that the ratio of Au to Gd(III), after cell internalization, remains constant for at least 24 hours. This implies that the DNA-Gd(III)-AuNP assembly did not undergo enzyme digestion over this time period which is consistent with previously published results using similar DNA-AuNP conjugates [Chithrani et al., Chan, Nano Lett. 6: 662 (2006)]. It was additionally noted that on average the cells internalize approximately 10⁵ Gd(III)-conjugates/cell, which is 2 orders of magnitude higher than citrate-stabilized AuNPs of the same size.

Confocal Laser Scanning Microscopy (CLSM).

NIH/3T3 and HeLa cells were grown to 30% confluence (using 100 μL working volumes) on 8 chamber Lab-Tek® II German coverglass systems (Nalge Nunc International, Naperville, Ill., USA). Cells were then incubated with 0.25 nM AuNP (20 nM Cy3) for 4.0 or 24 hours in phenol red free medium supplemented with serum (as described above). After AuNP incubation, cells were rinsed two times with DPBS followed by addition of 100 μL of fresh medium. Cells were then either prepared for imaging or incubated with fresh medium for 24 hours (at 37° C. and 5.0% CO₂, leached) followed by two DPBS rinses and addition of 100 μL of fresh medium and then prepared for imaging. Cells were prepared for imaging via labeling with 10 μM CellTracker® Green and 5 μM DAPI (Invitrogen, Carlsbad, Calif., USA) in complete medium for 30 minutes (at 37° C. and 5.0% CO₂), medium was then aspirated, cells were rinsed two times with DPBS, followed by addition of 100 μL of fresh medium. Images were acquired on a Zeiss LSM 510 inverted microscope (computer controlled using Zeiss Zen software) equipped with a mode-locked Mai Tai DeepSeee Ti:sapphire two-photon laser (Spectra Physics, Mountain View, Calif., USA). Specifically, DAPI was excited using 780 nm excitation wavelength (2-photon) at 8.4% laser power through a HFT KP 660 beamsplitter and imaged through a 435-485 nm IR bandpass filter (no pinhole). CellTrackert Green was excited using the 488 nm wavelength of an argon ion laser at 3.0% laser power through a HFT 488/543 beamsplitter and imaged with a PMT through a 500-550 nm IR bandpass filter (140 μm pinhole). Cy₃ (AuNPs) was excited using the 543 nm wavelength of an He/Ne laser at 4.0% laser power through a HFT 488/543 beamsplitter and imaged with a PMT through a 560-615 nm IR bandpass filter (140 μm pinhole). An Apochromat water immersion objective (40×, NA 1.2) was used for all measurements. All images were acquired at 1024×1024 resolution with 15 z-stack slices.

To confirm the intracellular accumulation and uptake efficiency of the DNA-Gd(III)-AuNPs, bimodal AuNP conjugates were synthesized by conjugating Cy₃ to the 5′ end of the DNA-Gd(III) strands [the ratio of optical to MR signal can be adjusted by altering the stoichiometry of the Cy3-labelled DNA-Gd(III) strands with non-labeled strands]. Specifically, NIH/3T3 and HeLa cells were labeled with 0.1-0.2 nM Cy3-DNA-Gd(III)-AuNPs for 24 hours, rinsed three times with DPBS, and imaged using a confocal laser scanning microscope (CLSM).

The fluorescence micrographs show that the Cy3-DNA-Gd(III)-AuNPs localize in small vesicles in the perinuclear region, which is consistent with previous reports that show AuNP conjugates are taken up through an endocytic mechanism [Chithrani et al., Nano Lett. 7: 1542 (2007)]. A second batch of cells was incubated under the same conditions and allowed to leach for 24 hours (media with contrast agent is replaced with fresh media after rinsing). During this time the cell number doubled, but the fluorescence signal persisted in essentially every cell.

Cell labeling efficiency was evaluated using analytical flow cytometry and showed that at 0.3 nM Cy3-DNA-Gd(III)-AuNP incubation concentration, 80% of the cells were labeled after 4.0 hours. In both NIH/3T3 and Hela cells, labeling reached 100% after a 24 hour incubation. Importantly, no evidence of cell toxicity or cell number variation was observed under any of the conditions tested using DNA-Gd(III)-AuNPs or DOTA-Gd(III).

This example demonstrated a multimodal, cell permeable MR contrast agent based upon polyvalent DNA-AuNPs. These particles exhibited excellent biocompatibility and stability, high Gd(III) loading, a greater than 50-fold increase in cell uptake compared to a clinically available contrast agent [DOTA-Gd(III)], and relatively high relaxivity. When modified with a fluorophore, the DNA-AuNPs can be used as multimodal imaging agents where fluorescence microscopy showed that the particles localize in the perinuclear region inside cells. Since AuNPs serve as CT contrast agents, these DNA-Gd(III)-AuNP conjugates have promise as multimodal imaging probes for MR, fluorescence, and CT. The library of available probes for cancer and biological cellular imaging is growing and the strategy presented in this work represents a promising new addition [Park et al., Bioorg. Me]. Chem. Lett. 18: 6135 (2008); Debouttiere et al., Adv. Funct. Mater 16: 2330 (2006); Moriggi et al., J. Am. Chem. Soc. 2009, 131: 10828 (2009); Smith et al., Adv. Drug Delivery Rev. 60: 1226 (2008); Alivisatos, Nat. Biotechnol. 22: 47 (2004); Xia, Nat. Mater. 7: 758 (2008); Kim et al., Nano Lett. 8: 3887 (2008)].

1. A composition comprising a nanoparticle functionalized with a polynucleotide, wherein the polynucleotide is conjugated to a contrast agent through a conjugation site.

2. The composition of claim 1, wherein the contrast agent is a paramagnetic compound, iodine or barium.

3. The composition of claim 1, wherein the paramagnetic compound is a paramagnetic gadolinium [Gd(III)] complex or a manganese chelate.

4. The composition of claim 1, wherein the polynucleotide comprises a homopolymer.

5. (canceled)

6. (canceled)

7. The composition of claim 1, wherein the polynucleotide further comprises a detectable marker.

8. The composition of claim 7, wherein the detectable marker is a fluorophore, an isotope, a mass tag, a quantum dot, or a metal.

9. (canceled)

10. (canceled)

11. (canceled)

12. (canceled)

13. The composition of claim 1, wherein the polynucleotide comprises one to ten conjugation sites or five conjugation sites.

14. (canceled)

15. (canceled)

16. (canceled)

17. The composition of claim 1 wherein the nanoparticle comprises about 50 to about 2.5×10⁶ contrast agents or about 500 to about 1×10⁶ contrast agents.

18. (canceled)

19. (canceled)

20. (canceled)

21. The composition of claim 1, further comprising a therapeutic agent.

22. A method of delivering a contrast agent to a cell comprising contacting the cell with the composition of claim 1 under conditions sufficient to deliver the contrast agent to the cell.

23. The method of claim 22 further comprising the step of detecting the contrast agent.

24. The method of claim 23 wherein the contrast agent is detected by detecting the detectable marker.

25. The method of claim 22 which is an imaging procedure.

26. (canceled)

27. The method of any one claim 22 wherein the cell is selected from the group consisting of a cancer cell, a stem cell, a T-cell, a β-islet cell and a neuron.

28. The method of claim 22 wherein delivery is in vivo.

29. The method of claim 27 wherein delivery is intravenous or intraarterial.

30. (canceled)

31. The method of claim 22 further comprising the step of identifying the cell to which the composition has been delivered.

32. The method of claim 22 wherein the delivery is in vitro.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The method of claim 22 further comprising delivery of an embolic agent.

46. (canceled)

47. (canceled)

48. (canceled)

49. (canceled)

50. A kit comprising the composition of claim 1.
